SEQUENCE LISTING (28 sequences)

SEQ ID NO: 1 - DNA, 6595 bp, synthetic construct
  source 1..6595: mol_type = other DNA
atgaaaattaaaactctggcaatcgttgttctgtcggctctgtccctcagttccacagcggctctggccgctgccacgacggttaatggtgggaccgttcactttaaaggggaagttgttaacgccgcttgcgcagttgatgcaggctctgttgatcaaaccgttcagttaggacaggttcgtaccgcatcgctggaccaggatggagcgaccagctctgctgtcggttttaacattcaactgaatgattgcgataccgatgttgcaagtaaagctgctgttgccttcttaggtacaacaattgacacgaatcataccaacgtactggctctgcaaagttctgctgcgggtagcgcaacaaacgttggtgtgcagatcctggataaaacagggactgcgttggctttggatggtgcgtcatttagtgcacaaaccaccctgaataacggaaccaacaccattccgttccaggcgcgttattatgccattggtgccgcaacccctggcgcagctaatgcggatgcgaccttcaaggttcagtatcaataacctacctaggttcagggacgtcattacgggcagggatgcccacccttgtgcgataaaaataacgatgaaaaggaagagattatttctgttagcgtcgttgctgccaatgtttgctctggccggaaataaatggaataccacgctgcccggcggaaatatgcaatttcagggcgtcattattgcggaagcttgccggattgaagccggtgataaacaaatgacggtcaatatggggcaaatcagcagtaaccggtttcatgcggtaggggaagatagctcaccggtgccttttgttatttatttacgggaatgtagcacggtggtgagtgaacgtgtgggtgtggcgtttcacggtgtcgcggatggtaaaaatccggatgtgctttccgtgggagaggggccagggatagccaccaatattggcgtagcgttatttgatgatgaaggaaacctcgtaccgattaatcgtcctccagcaaactggaaacggctttactcaggctctacttcgctacatttcatcgccaaatatcgtgctaccgggcgtcgggttactggcggcatcgccaatgcccaggcctggttctctttaacctatcagtaattgttcagcagataatgtgataacaggaacaggacagtgagtaataaaaacgtcaatgtaaggaaatcgcaggaaataacattctgcttgctggcaggtatcctgatgttcatggcaatggtggttgccggacgcgctgaagcgggagtggccttaggtgcgacccgcgtaatttatccggcagggcaaaaacaagtgcaacttgccgtgacaaataatgatgaaaatagcacctatttaattcaatcatgggtggaaaatgccgatggtgttaaggatggtcgttttatcgtgacgccacctctgtttgcgatgaagggaaaaaaagagaataccttgcgtattcttgatgcaacaaataaccaattgccacaggatcgggaaagtttattctggatgaatgttaaagcgattccgtcaatggataaatcaaaattgactgagaatacgctacagctcgcaattatcagccgcattaaactgtactatcgtccggctaaattagcgttgccacccgatcaggccgcagaaaaattaagatttcgtcgtagcgcgaattctctgacgctgattaacccgacaccctattacctgacggtaacagagttgaatgccggaactcgggttcttgaaaatgcattggtgcctccaatgggcgaaagcgcggttaaattgccttctgatgcaggaagcaatattacttaccgaacaataaatgattatggcgcacttacccccaaaatgacgggcgtaatggaataacgcagggggattttttcgcctgaataaaaagaattgactgccgggtgatttaaaccggaggaataatgtcatatctgaatttaagactttaccagcgaaacacacagtgcttgcatattcgtaagcatcgtttggctggtttttttgtccggctctttgttgcctgtgcttttgccgcacaggcacctttgtcatctgccgaactctattttaacccgcgttttttagcggatgatccccaggctgtggctgatttatcgcgttttgagaatgggcaagaattaccgccagggacgtatcgtgtcgatatctatttgaataatggttatatggcaacgcgtgatgtcacatttaatacgggcgacagcgaacaagggattgttccctgcctgacacgcgcgcaacttgccagtatggggctgaatacggcttctgtctccggtatgaatctgctggcggatgatgcctgcgtgccattaacctcaatgatccatgacgctactgcgcatttagatgttggtcagcagcgactcaacctgacgatccctcaggcatttatgagtaatcgcgcgcgtggttatattcctcctgagttatgggatcccggtattaatgccggattgctcaattataatttcagcggaaatagtgtacagaatcgaattgggggtaacagccattatgcatatttaaacctacagagtgggttaaatattggtgcgtggcgtttacgcgacaataccacctggagttataacagtagcgacagttcatcaggtagcaaaaataaatggcagcatatcaatacctggcttgagcgagacataattccgttacgttcccggctgacgttgggtgatggttatactcagggcgatattttcgatggtattaactttcgcggcgcacaattggcctcagatgacaatatgttacccgatagccaaagaggatttgccccggtgatccacggtattgctcgtggtactgcacaggtcactattaaacaaaatgggtatgacatttataatagtacggtgccaccggggccttttaccatcaacgatatctatgccgcaggtaatagtggtgacttgcaggtaacgattaaagaggctgacggcagtacgcagatttttaccgtaccctattcgtcagtcccgcttttgcagcgtgaagggcatacacgttattccattacggcaggagaataccgtagtggaaatgcgcaacaggaaaaacctcgctttttccagagtacattactccacggccttccggctggctggacaatatatggtggaacgcaactggcggatcgttatcgtgcttttaatttcggtatcgggaaaaacatgggggcactgggcgctctgtctgtggatatgacgcaggctaattccacacttcccgatgacagtcagcatgacggacaatcggtgcgttttctctataacaaatcgctcaatgagtcaggcacgaatattcagttagtgggttaccgttattcgaccagtgggtattttaatttcgctgatacaacatacagtcgaatgaatggctacaacatcgaaacacaggatggagttattcaggttaagccgaaattcaccgactattacaatctcgcttataacaaacgcgggaaattacaactcaccgttactcagcaactcgggcgcacatcaacactgtatttgagtggtagccatcaaacttattggggaacgagtaatgtcgatgagcaattccaggctggattaaatactgcgttcgaagatattaactggacgctcagctatagcctgacgaaaaacgcctggcaaaaaggacgggatcagatgttagcgcttaacgtcaatattcctttcagccactggctgcgttctgacagtaaatctcagtggcgacatgccagtgccagttacagcatgtcacacgatctcaacggtcggatgaccaatctggctggtgtatacggtacgttgctggaagacaacaacctcagctatagcgtgcaaaccggctatgccgggggaggcgatggtaatagcggaagtacaggctacgccacgctgaattatcgcggtggttacggcaatgccaatatcggttacagccatagcgatgatattaagcagctctattacggagtcagcggtggggtactggctcatgccaatggcgtaacgctggggcagccgttaaacgatacggtggtgcttgttaaagcgcctggcgcaaaagatgcaaaagtcgaaaaccagacgggggtgcgtaccgactggcgcggttatgccgtgctgccttatgccactgaatatcgggaaaacagagtagcgctggataccaataccctggctgataacgtcgatttagataacgcggtcgctaacgttgttcccacccgtggggcgatcgtgcgagcagagtttaaagcgcgcgttgggataaaactgctcatgacgctaacccacaataataagccgctgccgtttggggcgatggtgacatcagagagtagccagagtagcggcattgttgcggataatggtcaggtttacctcagcggaatgcctttagcgggaaaagtgcaggtgaaatggggagaagaggaaaatgctcactgtgtcgccaattatcaactgccaccagagagtcagcagcagttattaacccagctatcagctgaatgtcgttaagggggcgtgatgagaaacaaacctttttatcttctttgcgcttttttgtggctggcggtaagtcacgctttggctgcggatagcacgattactatccgcggctatgtcagagataatggctgtagtgtggccgctgaatcaaccaattttactgttgatctgatggaaaacgcggcgaagcaatttaacaacattggtgcgacgactcctgtcgttccatttcgtattttgctgtcaccctgtggtaacgctgtttctgccgtaaaggttgggtttactggcgttgcagatagccacaatgccaacctgcttgcacttgaaaatacggtgtcagcggctgcgggactgggaatacagcttctgaatgagcagcaaaatgagataccccttaatgccccatcgtccgcgatttcgtggacgaccctgacgccgggtaaaccaaatacgttgaatttttacgcccggctaatggcgacacaggtgcctgtcactgcggggcatatcaatgccacggctaccttcactcttgaatatcagtaactggagatgctcatgaaatggcgcaaacgtgggtatttattggcggcaatgttggcgttcgcaagtgcgacgatacaggcagccgatgtcaccatcacggttaacggtaaggtcgtcgccaaaccgtgtacggtttccaccaccaatgccacggtagatctcggcgatctttattctttcagtctgatgtctgccggggcggcatcggcctggcatgatgttgcgcttgagttgactaattgtccggtgggaacgtcgagggtcactgccagcttcagcggggcagccgacagtaccggatattataaaaatcaggggaccgcgcaaaacatccagttagagctacaggatgacagtggcaacacattgaatactggcgcaaccaaaacagttcaggtggatgattcctcacaatcagcgcacttcccgttacaggtcagagcattgaccgtaaatggtggagccactcagggaaccattcaggcagtgattagcatcacctatacctacagctgaacccaaagagatgattgtaatgaaacgagttattaccctgtttgctgtactgctgatgggctggtcggtaaatgcctggtcattcgcctgtaaaaccgccaatggtaccgcaatccctattggcggtggcagcgccaatgtttatgtaaaccttgcgcctgccgtgaatgtggggcaaaacctggtcgtagatctttcgacgcaaatcttttgccataacgattacccagaaaccattacagactatgtcacactgcaacgaggttcggcttatggcggcgtgttatctagtttttccgggaccgtaaaatataatggcagtagctatcctttccctactaccagcgaaacgccgcgggttgtttataattcgagaacggataagccgtggccggtggcgctttatttgacgccggtgagcagtgcggggggagtggcgattaaagctggctcattaattgccgtgcttattttgcgacagaccaacaactataacagcgatgatttccagtttgtgtggaatatttacgccaataatgatgtggtggtgcccactggcggctgtgatgcttctgctcgtgatgtcaccgttactctgccggactaccctggttcagtgccgattcctcttaccgtttattgtgcgaaaagccaaaacctggggtattacctatccggcacaaccgcagatgcgggcaactcgattttcaccaataccgcgtcgttttcacccgcgcagggcgtcggcgtacagttgacgcgcaacggtacgattattccagcgaataacacggtatcgttaggagcagtagggacttcggcggtaagtctgggattaacggcaaattacgcacgtaccggagggcaggtgactgcagggaatgtgcaatcgattattggcgtgacttttgtttatcaataa

SEQ ID NO: 2 - DNA, 35 bp, synthetic construct
  source 1..35: mol_type = other DNA
ttgacagctagctcagtcctagggattgtgctagc

SEQ ID NO: 3 - DNA, 7030 bp, synthetic construct
  source 1..7030: mol_type = other DNA
attggggccaaactgtccatatcataaataagttacgtattttttctcaagcataaaaatattaaaaaacgacaaaaagcatctaactgtttgatatgtaaattatttctattgtaaattaatttcacatcacctccgctatatgtaaagctaacgtttctgtggctcgacgcatcttcctcattcttctctccaaaaaccacctcatgcaatataaacatctataaataaagataacaatagaatattaagccaacaaataaactgaaaaagtttgtccgcgatgctttcctctatgagtcaaaatggccccaaatgtttcatcttttgggggaaaactgtgcagtgttggcagtcaaactcgtttacaaaacaaagtgtacagaacgactgcccatgtcgatttagaaatagttttttgaaaggaaagcagcatgaaaattaaaactctggcaatcgttgttctgtcggctctgtccctcagttctacagcggctctggccgctgccacgacggttaatggtgggaccgttcactttaaaggggaagttgttaacgccgcttgcgcagttgatgcaggctctgttgatcaaaccgttcagttaggacaggttcgtaccgcatcgctggcacaggaaggagcaaccagttctgctgtcggttttaacattcagctgaatgattgcgataccaatgttgcatctaaagccgctgttgcctttttaggtacggcgattgatgcgggtcataccaacgttctggctctgcagagttcagctgcgggtagcgcaacaaacgttggtgtgcagatcctggacagaacgggtgctgcgctgacgctggatggtgcgacatttagttcagaaacaaccctgaataacggaaccaataccattccgttccaggcgcgttattttgcaaccggggccgcaaccccgggtgctgctaatgcggatgcgaccttcaaggttcagtatcaataacctacccaggttcagggacgtcattacgggcagggatgcccacccttgtgcgataaaaataacgatgaaaaggaagagattatttctattagcgtcgttgctgccaatgtttgctctggccggaaataaatggaataccacgttgcccggcggaaatatgcaatttcagggcgtcattattgcggaaacttgccggattgaagccggtgataaacaaatgacggtcaatatggggcaaatcagcagtaaccggtttcatgcggttggggaagatagcgcaccggtgccttttgttattcatttacgggaatgtagcacggtggtgagtgaacgtgtaggtgtggcgtttcacggtgtcgcggatggtaaaaatccggatgtgctttccgtgggagaggggccagggatagccaccaatattggcgtagcgttgtttgatgatgaaggaaacctcgtaccgattaatcgtcctccagcaaactggaaacggctttattcaggctctacttcgctacatttcatcgccaaatatcgtgctaccgggcgtcgggttactggcggcatcgccaatgcccaggcctggttctctttaacctatcagtaattgttcagcagataatgtgataacaggaacaggacagtgagtaataaaaacgtcaatgtaaggaaatcgcaggaaataacattctgcttgctggcaggtatcctgatgttcatggcaatgatggttgccggacgcgctgaagcgggagtggccttaggtgcgactcgcgtaatttatccggcagggcaaaaacaagagcaacttgccgtgacaaataatgatgaaaatagtacctatttaattcaatcatgggtggaaaatgccgatggtgtaaaggatggtcgttttatcgtgacgcctcctctgtttgcgatgaagggaaaaaaagagaataccttacgtattcttgatgcaacaaataaccaattgccacaggaccgggaaagtttattctggatgaacgttaaagcgattccgtcaatggataaatcaaaattgactgagaatacgctacagctcgcaattatcagccgcattaaactgtactatcgcccggctaaattagcgttgccacccgatcaggccgcagaaaaattaagatttcgtcgtagcgcgaattctctgacgctgattaacccgacaccctattacctgacggtaacagagttgaatgccggaacccgggttcttgaaaatgcattggtgcctccaatgggcgaaagcacggttaaattgccttctgatgcaggaagcaatattacttaccgaacaataaatgattatggcgcacttacccccaaaatgacgggcgtaatggaataacgcagggggaatttttcgcctgaataaaaagaattgactgccgggtgattttaagccggaggaataatgtcatatctgaatttaagactttaccagcgaaacacacaatgcttgcatattcgtaagcatcgtttggctggtttttttgtccgactcgttgtcgcctgtgcttttgccgcacaggcacctttgtcatctgccgacctctattttaatccgcgctttttagcggatgatccccaggctgtggccgatttatcgcgttttgaaaatgggcaagaattaccgccagggacgtatcgcgtcgatatctatttgaataatggttatatggcaacgcgtgatgtcacatttaatacgggcgacagtgaacaagggattgttccctgcctgacacgcgcgcaactcgccagtatggggctgaatacggcttctgtcgccggtatgaatctgctggcggatgatgcctgtgtgccattaaccacaatggtccaggacgctactgcgcatctggatgttggtcagcagcgactgaacctgacgatccctcaggcatttatgagtaatcgcgcgcgtggttatattcctcctgagttatgggatcccggtattaatgccggattgctcaattataatttcagcggaaatagtgtacagaatcggattgggggtaacagccattatgcatatttaaacctacagagtgggttaaatattggtgcgtggcgtttacgcgacaataccacctggagttataacagtagcgacagatcatcaggtagcaaaaataaatggcagcatatcaatacctggcttgagcgagacataataccgttacgttcccggctgacgctgggtgatggttatactcagggcgatattttcgatggtattaactttcgcggcgcacaattggcctcagatgacaatatgttacccgatagtcaaagaggatttgccccggtgatccacggtattgctcgtggtactgcacaggtcactattaaacaaaatgggtatgacatttataatagtacggtgccaccggggccttttaccatcaacgatatctatgccgcaggtaatagtggtgacttgcaggtaacgatcaaagaggctgacggcagcacgcagatttttaccgtaccctattcgtcagtcccgcttttgcaacgtgaagggcatactcgttattccattacggcaggagaataccgtagtggaaatgcgcagcaggaaaaaacccgctttttccagagtacattactccacggccttccggctggctggacaatatatggtggaacgcaactggcggatcgttatcgtgcttttaatttcggtatcgggaaaaacatgggggcactgggcgctctgtctgtggatatgacgcaggctaattccacacttcccgatgacagtcagcatgacggacaatcggtgcgttttctctataacaaatcgctcaatgaatcaggcacgaatattcagttagtgggttaccgttattcgaccagcggatattttaatttcgctgatacaacatacagtcgaatgaatggctacaacatcgaaacacaggacggagttattcaggttaagccgaaattcaccgactattacaacctcgcttataacaaacgcgggaaattacaactcaccgttactcagcaactcgggcgcacatcaacactgtatttgagtggtagccatcaaacttattggggaacgagtaatgtcgatgagcaattccaggctggattaaatactgcgttcgaagatatcaactggacgctcagctatagcctgacgaaaaacgcctggcaaaaaggacgggatcagatgttagcgcttaacgtcaatattcctttcagccactggctgcgttctgacagtaaatctcagtggcgacatgccagtgccagctacagcatgtcacacgatctcaacggtcggatgaccaatctggctggtgtatacggtacgttgctggaagacaacaacctcagctatagcgtgcaaaccggctatgccgggggaggcgatggaaatagcggaagtacaggctacgccacgctgaattatcgcggtggttacggcaatgccaatatcggttacagccatagcgatgatattaagcagctctattacggagtcagcggtggggtactggctcatgccaatggcgtaacgctggggcagccgttaaacgatacggtggtgcttgttaaagcgcctggcgcaaaagatgcaaaagtcgaaaaccagacgggggtgcgtaccgactggcgtggttatgccgtgctgccttatgccactgaatatcgggaaaatagagtggcgctggataccaataccctggctgataacgtcgatttagataacgcggttgctaacgttgttcccactcgtggggcgatcgtgcgagcagagtttaaagcgcgcgttgggataaaactgctcatgacgctgacccacaataataagccgctgccgtttggggcgatggtgacatcagagagtagccagagtagcggcattgttgcggataatggtcaggtttacctcagcggaatgcctttagcgggaaaagttcaggtgaaatggggagaagaggaaaatgctcactgtgtcgccaattatcaactgccaccagagagtcagcagcagttattaacccagctatcagctgaatgtcgttaagggggcgtgatgagaaacaaacctttttatcttctgtgcgcttttttgtggctggcggtgagtcacgctttggctgcggatagcacgattactatccgcggctatgtcagggataacggctgtagtgtggccgctgaatcaaccaattttactgttgatctgatggaaaacgcggcgaagcaatttaacaacattggcgcgacgactcctgttgttccatttcgtattttgctgtcaccctgtggtaatgccgtttctgccgtaaaggttgggtttactggcgttgcagatagccacaatgccaacctgcttgcacttgaaaatacggtgtcagcggcttcgggactgggaatacagcttctgaatgagcagcaaaatcaaataccccttaatgctccatcgtccgcgctttcgtggacgaccctgacgccgggtaaaccaaatacgctgaatttttacgcccggctaatggcgacacaggtgcctgtcactgcggggcatatcaatgccacggctaccttcactcttgaatatcagtaactggagatgctcatgaaatggtgcaaacgtgggtatgtattggcggcaatattggcgctcgcaagtgcgacgatacaggcagccgatgtcaccatcacggtgaacggtaaggtcgtcgccaaaccgtgtacggtttccaccaccaatgccacggttgatctcggcgatctttattctttcagtcttatgtctgccggggcggcatcggcctggcatgatgttgcgcttgagttgactaattgtccggtgggaacgtcgagggtcactgccagcttcagcggggcagccgacagtaccggatattataaaaaccaggggaccgcgcaaaacatccagttagagctacaggatgacagtggcaacacattgaatactggcgcaaccaaaacagttcaggtggatgattcctcacaatcagcgcacttcccgttacaggtcagagcattgacagtaaatggcggagccactcagggaaccattcaggcagtgattagcatcacctatacctacagctgaacccgaagagatgattgtaatgaaacgagttattaccctgtttgctgtactgctgatgggctggtcggtaaatgcctggtcattcgcctgtaaaaccgccaatggtaccgctatccctattggcggtggcagcgccaatgtttatgtaaaccttgcgcccgtcgtgaatgtggggcaaaacctggtcgtggatctttcgacgcaaatcttttgccataacgattatccggaaaccattacagactatgtcacactgcaacgaggctcggcttatggcggcgtgttatctaatttttccgggaccgtaaaatatagtggcagtagctatccatttcctaccaccagcgaaacgccgcgcgttgtttataattcgagaacggataagccgtggccggtggcgctttatttgacgcctgtgagcagtgcgggcggggtggcgattaaagctggctcattaattgccgtgcttattttgcgacagaccaacaactataacagcgatgatttccagtttgtgtggaatatttacgccaataatgatgtggtggtgcctactggcggctgcgatgtttctgctcgtgatgtcaccgttactctgccggactaccctggttcagtgccaattcctcttaccgtttattgtgcgaaaagccaaaacctggggtattacctctccggcacaaccgcagatgcgggcaactcgattttcaccaataccgcgtcgttttcacctgcacagggcgtcggcgtacagttgacgcgcaacggtacgattattccagcgaataacacggtatcgttaggagcagtagggacttcggcggtgagtctgggattaacggcaaattatgcacgtaccggagggcaggtgactgcagggaatgtgcaatcgattattggcgtgacttttgtttatcaataa

SEQ ID NO: 4 - DNA, 314 bp, synthetic construct
  source 1..314: mol_type = other DNA
ttggggccattttgactcatagaggaaagcatcgcgcacaaactttttcagtttatttgttggcttaatgttctatagttatctttatttgcagttttttatattgcatgaggtggtttttggagagaagaatgaggaagttgcgtcgagctacagaaacgttagctttacatatagcggaggtgatgtgaaattaatttacaagagaaataatttacatatcaaacagttagatgctttttgtcgttttttaatatttttatgcttgagaaaaaatacgtaacttatttatgatatagacagtttggccccaa

SEQ ID NO: 5 - DNA, 314 bp, synthetic construct
  source 1..314: mol_type = other DNA
ttagatctcttttgactcatagaggaaagcatcgcgcacaaactttttcagtttatttgttggcttaatgttctatagttatctttatttgcagttttttatattgcatgaggtggtttttggagagaagaatgaggaagttgcgtcgagctacagaaacgttagctttacatatagcggaggtgatgtgaaattaatttacaagagaaataatttacatatcaaacagttagatgctttttgtcgttttttaatatttttatgcttgagaaaaaatacgtaacttatttatgatatagacagtttggccccaa

SEQ ID NO: 6 - DNA, 314 bp, synthetic construct
  source 1..314: mol_type = other DNA
ttggggccattttgactcatagaggaaagcatcgcggacaaactttttcagtttatttgttggcttaatattctattgttatctttatttatagatgtttatattgcatgaggtggtttttggagagaagaatgaggaagatgcgtcgagccacagaaacgttagctttacatatagcggaggtgatgtgaaattaatttacaatagaaataatttacatatcaaacagttagatgctttttgtcgttttttaatatttttatgcttgagaaaaaatacgtaacttatttatgatatggacagtttggccccaa

SEQ ID NO: 7 - DNA, 314 bp, synthetic construct
  source 1..314: mol_type = other DNA
ttagatctcttttgactcatagaggaaagcatcgcggacaaactttttcagtttatttgttggcttaatattctattgttatctttatttatagatgtttatattgcatgaggtggtttttggagagaagaatgaggaagatgcgtcgagccacagaaacgttagctttacatatagcggaggtgatgtgaaattaatttacaatagaaataatttacatatcaaacagttagatgctttttgtcgttttttaatatttttatgcttgagaaaaaatacgtaacttatttatgatatggacagtttggccccaa

SEQ ID NO: 8 - DNA, 539 bp, synthetic construct
  source 1..539: mol_type = other DNA
atgctaaagtatgaggagagaaaattgaataatttaacgttgtcttcgttttcaaaagttggtgtgtcgaatgatgcccgactttatattgctaaggaaaatactgataaggcatatgttgcgcctgaaaaattttcgtcaaaagtattaacctggcttggaaaaatgccgttatttaaaaacactgaagtggtgcaaaaacatacggaaaatatcagagtacaggaccaaaagattttacagacatttctccatgcactaacggaaaaatatggggaaacagcggttaatgacgcactgttaatgtcccgtataaatatgaacaaaccccttacccaacgtttagcagtgcagatcacggagtgtgtaaaagctgctgacgaagggtttataaaccttattaagagcaaggataatgttggtgtcaggaatgccgctttagtcataaaaggcggcgatacaaaagtggcagaaaaaaataacgatgttggagcagaaagtaagcaacctttactcgatatcgcgctaaagggacttaa

SEQ ID NO: 9 - DNA, 150 bp, synthetic construct
  source 1..150: mol_type = other DNA
atgccagtcactttaagcttcggtaatcatcaaaattatacgcttaatgaaagtcggcttgctcatctgttaagcgcagataaagaaaaagcaatccatatggggggttgggataaagtccaggatcattttagagcggaaaaaaaggac

SEQ ID NO: 10 - DNA, 540 bp, synthetic construct
  source 1..540: mol_type = other DNA
atggttacaagtgtaaggactcagccccccgtcataatgccaggtatgcagaccgagatcaaaacgcaggccacgaatcttgcggcgaatctttccgcagtcagagaaagtgccacagcgacgctgtcaggggaaattaaaggcccgcaactggaagattttcccgcgctgatcaaacaggcgagtctggatgcattgtttaaatgcgggaaagacgctgaggcgttaaaagaagtttttaccaattcaaataatgtcgccggtaagaaagcgataatggagtttgccgggctctttcgttcagcgctcaacgccaccagtgattctcctgaggcgaagacgctactgatgaaggtgggggcagagtataccgcgcaaatcataaaagatggcctgaaagaaaagtcagcttttgggccatggctgccagaaacaaagaaagcggaagcgaagctggaaaacctggaaaagcagctgttagatattatcaaaaataacactggcggtgaattaagtaaattatcgacgaatcttgttatg

SEQ ID NO: 11 - DNA, 600 bp, synthetic construct
  source 1..600: mol_type = other DNA
atgtttaatattactaatatacaatctacggcaaggcatcaaagtattagcaatgaggcctcaacagaggtgcctttaaaagaagagatatggaataaaataagtgcctttttctcttcagaacatcaggttgaagcacaaaactgcatcgcttatctttgtcatccacctgaaaccgcctcgccagaagagatcaaaagcaagtttgaatgtttaaggatgttagctttcccggcgtatgcggataatattcagtatagtagaggaggggcagaccaatactgtattttgagtgaaaatagtcaggaaattctgtctatagtttttaatacagagggctataccgttgagggagggggaaagtcagtcacctatacccgtgtgacagaaagcgagcaggcgagtagcgcttccggctccaaagatgctgtgaattatgagttaatctggtctgagtgggtaaaagaggcgccagcgaaagaggcagcaaatcgtgaagaagccgtacaacggatgcgtgactgcctgaaaaataataagacggaacttcgtctgaaaatattaggacttaccactatacctgcctatattcctgagcag

SEQ ID NO: 12 - DNA, 300 bp, synthetic construct
  source 1..300: mol_type = other DNA
atgaagatatcatcaggcgcaattaatttttctactattcctaaccaggttaaaaaattaattacctctattcgtgaacatacgaaaaacgggctcacctcaaaaataaccagtgttaaaaacacgcatacatctttaaatgaaaaatttaaaacaggaaaggactcaccgattgagttcgcgttaccacaaaaaataaaagacttctttcagccgaaagataaaaacaccttaaacaaaacattgattactgttaaaaatattaaagatacaaataatgcaggcaagaaaaatatttca

SEQ ID NO: 13 - DNA, 540 bp, synthetic construct
  source 1..540: mol_type = other DNA
atgcaaatacagagcttctatcactcagcttcactaaaaacccaggaggcttttaaaagcctacaaaaaaccttatacaacggaatgcagattctctcaggccagggcaaagcgccggctaaagcgcccgacgctcgcccggaaattattgtcctgcgagaacccggcgcgacatgggggaattatctacagcatcagaaggcgtctaaccactcgctgcataacctctataacttacagcgcgatcttcttaccgtcgcggcaaccgttctgggtaaacaagacccggttctaacgtcaatggcaaaccaaatggagttagccaaagttaaagcggaccggccagcaacaaaacaagaagaagccgcggcaaaagcattgaagaaaaatcttatcgaacttattgcagcacgcactcagcagcaggatggcttacctgcaaaagaagctcatcgctttgcggcagtagcgtttagagatgctcaggtcaagcagcttaataaccagccctggcaaaccataaaaaatacactcacgcat

SEQ ID NO: 14 - DNA, 540 bp, synthetic construct
  source 1..540: mol_type = other DNA
atgttaattagtaatgtgggaataaatcccgccgcttatttaaataatcattctgttgagaatagttcacagacagcttcgcaatccgttagcgctaaagatattctgaatagtattggtattagcagcagtaaagtcagtgacctggggttgagtcctacactgagcgcgcctgcgccaggggtattaacgcaaacccccggaacgatcacgtcctttttaaaagccagtattcaaaataccgacatgaatcaggatttgaatgctctggcaaataatgtcacgactaaagcgaatgaggttgtgcaaacccagttacgcgagcagcaggcagaagtcggaaagttttttgatattagcggaatgtcttccagtgccgttgcgctgttggctgccgcgaatacgttaatgctgacgttgaaccaggctgatagcaaactgtctggtaagttgtcattagtcagttttgatgcagctaaaacgacggcaagctccatgatgcgcgaagggatgaatgcgttgtccggtagtatttcccag

SEQ ID NO: 15 - DNA, 300 bp, synthetic construct
  source 1..300: mol_type = other DNA
atgatattttcggtgcaggagctatcatgtggagggaaaagtatgctaagtcctacgactcgtaatatgggggcgagtttatcgcctcagcctgacgtcagcggggagctaaacaccgaagcattgacctgtattgttgagcgtctggaaagtgaaattatagatggcagctggattcatatcagttacgaggaaaccgatctcgaaatgatgccttttcttgttgcacaggccaataagaagtatccagagttaaatcttaaatttgttatgtcagtccatgagcttgtttcctctata

SEQ ID NO: 16 - DNA, 867 bp, synthetic construct
  source 1..867: mol_type = other DNA
atgaatttagatggtgttagaccatactgtagaatagtcaataaaaagaatgaaagcatatcagatattgcatttgcacatataataaaaagggtaaaaaattcatcatgtactcacccaaaagcagcattggtttttttaggagagaaaggtttttgtgatagcaatgatgttctatctattatgggacaacaaataccaagagtatttaagaacaagatgttatatgattatgtttttaaaaatgaaaaaagtaaaaatgattttctaaaaatggctgaatcatggctaccacagagtgaaccaatagtaataaataatgatgatgacgcattgaatgctgctgcttatttttctgtaaaaaaagcgaaaataaaaacagtaaacgatactgattttaaagagtataataaggtttatattcttgggcacggtagtcctggttctcatcaattaggccttggttcggaacttattgatgtacaaacaatcatttcaagaatgaaagactgtggtattctaaatgtgaaagatatccgttttacttcatgcggctccgctgataaagtggctcctaaaaattttaacaatgcccctgctgaaagtctttcttgtatccttaactctctgcctttttttaaggaaaaagaatctttgctagagcagataaaaaaacaccttgaaaacgatgagtcattgagtgatggtctaaaaatatccggctatcatggatatggagttcactatggtcaagagctttttccctactcacattatcgttcaacttcaattcctgctgatccggagcatacagtaaaaagaagctctcagaaaaagacttttattattaataaagaactggattag

SEQ ID NO: 17 - DNA, 360 bp, synthetic construct
  source 1..360: mol_type = other DNA
gtgactaacataacactatccacccagcactacagaatccatagaagtgacgttgaaccagtaaaagaaaaaacaacggagaaggacatttttgcaaaaagtattactgccgttagaaatagctttatcagcctgtcgacgagtctgtcagatcgttttagcctgcatcaacaaacagacataccgactacccattttcatcgtgggaacgcttctgagggtagggcggtattaaccagtaaaactgttaaagattttatgctgcaaaagctcaatagtctggatatcaaaggtaatgcgagtaaagatccggcctatgctcgtcagacatgcgaagccatattatcagccgtgtacagt

SEQ ID NO: 18 - DNA, 180 bp, synthetic construct
  source 1..180: mol_type = other DNA
atgttaccgataaataataacttttcattgccccaaaattctttttataacactatttccggtacatatgctgattacttttcagcatgggataaatgggaaaaacaagcgctccccggtgaagagcgtgatgaggctgtctcccgacttggtggaggtggctccggcggcggaggttcc

SEQ ID NO: 19 - DNA, 180 bp, synthetic construct
  source 1..180: mol_type = other DNA
atgaaaatacctgaagcagtaaatcatattaatgttcagaacaatattgatcttgttgatggaaaaataaatccgaacaaagatacaaaagcattacaaaaaaacatatcatgcgtaacaaactcatcctcttctggcataagtgaaaaaggtggaggtggctccggcggcggaggttcc

SEQ ID NO: 20 - DNA, 180 bp, synthetic construct
  source 1..180: mol_type = other DNA
atgaaaataacatctaccattattcaaacaccttttccatttgagaataataattctcatgctggcatagtaacggagcccattctcggtaagttaataggtcaggggtcgacagcagaaatctttgaagatgtgaatgattcatctgctggtggaggtggctccggcggcggaggttcc

SEQ ID NO: 21 - DNA, 180 bp, synthetic construct
  source 1..180: mol_type = other DNA
atgaaaccgatcaacaatcattctttttttcgttccctttgtggcttatcatgtatatctcgtttatcggtagaagaacagtgtaccagagattaccaccgcatctgggatgactgggctagggaaggaacaacaacagaaaatcgcatcggtggaggtggctccggcggcggaggttcc

SEQ ID NO: 22 - DNA, 180 bp, synthetic construct
  source 1..180: mol_type = other DNA
atgttctctgtaaataatacacactcatcagtttcttgctccccctctattaactcaaactcaaccagtaatgaacattatctgagaatcctgactgaatgggaaaagaactcttctcccggggaagagcgaggcattgcttttaacagaggtggaggtggctccggcggcggaggttcc

SEQ ID NO: 23 - DNA, 162 bp, synthetic construct
  source 1..162: mol_type = other DNA
atgcatatccaatcgcttcaacagtccccgtcttttgctgttgaattgcaccaggcagccagtgggcgtttaggccagattgaagcacggcaagtggccaccccgagcgaagcgcagcagctggcgcagcgccaggatgcgccaaaaggtgagggcctgctg

SEQ ID NO: 24 - DNA, 150 bp, synthetic construct
  source 1..150: mol_type = other DNA
atgaaaatctcaagctttatttcgacttcactgccactgccgacgtctgtgtccggttcgagcagcgttggcgaaatgagtgggcgctccgtctctcagcaaaccagtgatcagtatgccaacaatttagcgggccgtaccgagagcccg

SEQ ID NO: 25 - protein, 88 aa, Escherichia coli
MALLDFFLSRKKNTANIAKERLQIIVAERRRSDAEPHYLPQLRKDILEVICKYVQIDPEMVTVQLEQKDGDISILELNVTLPEAEELK

SEQ ID NO: 26 - protein, 231 aa, Escherichia coli
MSNTPIELKGSSFTLSVVHLHEAEPKVIHQALEDKIAQAPAFLKHAPVVLNVSALEDPVNWSAMHKAVSATGLRVIGVSGCKDAQLKAEIEKMGLPILTEGKEKAPRPAPTPQAPAQNTTPVTKTRLIDTPVRSGQRIYAPQCDLIVTSHVSAGAELIADGNIHVYGMMRGRALAGASGDRETQIFCTNLMAELVSIAGEYWLSDQIPAEFYGKAARLQLVENALTVQPLN

SEQ ID NO: 27 - protein, 270 aa, Escherichia coli
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 28 - protein, 164 aa, Escherichia coli
MPLTPNDIHNKTFTKSFRGYDEDEVNEFLAQVRKDYEIVLRKKTELEAKVNELDERIGHFANIEETLNKSILVAQEAAEDVKRNSQKEAKLIVREAEKNADRIINESLSKSRKIAMEIEELKKQSKVFRTRFQMLIEAQLDLLKNDDWDHLLEYEVDAVFEEKE

(19) United States
(12) Patent Application Publication
(10) Pub. No.: US 2025/0375386 A1
(43) Pub. Date: Dec. 11, 2025

(54) ADAS COMPRISING TYPE 1 PILI

(71) Applicant: Flagship Pioneering Innovations VI, LLC, Cambridge, MA (US)

(72) Inventors: David Barry KOLESKY, Cambridge, MA (US); Caitlin Nicole SPAULDING, Boston, MA (US); Troy Patrick HUBBARD, Somerville, MA (US); Charles Martin FOX, Boston, MA (US); Kimberly A. HOMAN, Mill Valley, CA (US)

(21) Appl. No.: 18/877,551

(22) PCT Filed: Jun. 23, 2023
(86) PCT No.: PCT/US2023/068993

§ 371 Date: Dec. 20, 2024

Related U.S. Application Data
(60) Provisional application No. 63/354,979, filed on Jun. 23, 2022.

(51) Int. Cl. A61K 9/50; A61K 9/51; A61K 47/69; C07K 14/245; C12N 1/20; C12N 15/70; C12R 1/19

(52) U.S. Cl. CPC A61K 9/5052; A61K 9/5192; A61K 47/6925; C07K 14/245; C12N 1/20; C12N 15/70; C12R 2001/19

(57) ABSTRACT

Provided herein are achromosomal dynamic active systems comprising a Type 1 pilus (TIP) and methods of making and using the same.

### CROSS-REFERENCE TO RELATED APPLICATIONS

This application claims benefit of priority to U.S. Provisional Application No. 63/354,979, filed Jun. 23, 2022, which is herein incorporated by reference in its entirety.

### SEQUENCE LISTING

The instant application contains a Sequence Listing which has been submitted electronically in XML format and is hereby incorporated by reference in its entirety. Said XML copy, created on Jun. 20, 2023, is named 51296-056WO2_Sequence_Listing_6_20_23 and is 46,583 bytes in size.

### FIELD OF THE INVENTION

Provided herein are achromosomal dynamic active systems comprising a Type 1 pilus (T1P) and methods of making and using the same.

### BACKGROUND

A need exists for delivery vectors capable of targeting mammalian cells and delivering biological agents, compositions containing such delivery vectors, and associated methods of delivering said vectors to cells, thereby modulating biological systems including mammalian cells and organisms. In particular, there is a need for delivery vectors having tropism to target cells.

### SUMMARY OF THE INVENTION

In one aspect, the disclosure features a preparation comprising a plurality of achromosomal dynamic active systems (ADAS) derived from parent bacterial cells genetically engineered to constitutively express a type 1 pilus (T1P), wherein the plurality of ADAS binds a target cell via the T1P. In one aspect, the disclosure features a plurality of achromosomal dynamic active systems (ADAS) comprising a type 1 pilus (T1P), wherein the ADAS are derived from parent bacterial cells that constitutively express the components of the T1P.

In some embodiments, the parent cells comprise a modified fimS promoter that is operably linked to, and directs constitutive expression of, the components of the T1P.

In some embodiments, the components of the T1P are encoded by a fim operon.

In some embodiments, the parent cells comprise a modified fimS promoter operably linked to the fim operon, wherein the modified fimS promoter comprises a mutation at a recombinase cleavage site that prevents recombination of the fimS promoter into an ‘OFF’ orientation.

In some embodiments, the parent cells express the components of the T1P at a level that is at least 1.5-fold greater than a level observed in an unmodified parent cell.

In some embodiments, the ADAS comprise the T1P at a level that is at least 1.5-fold greater than a level observed in a plurality of ADAS produced from unmodified parent cells.

In some embodiments, the proportion of the plurality of ADAS comprising a T1P is increased relative to a plurality of ADAS produced by parent cells that do not constitutively express the components of a T1P.

In some embodiments, the parent cells comprise an endogenous fim operon.

In some embodiments, the parent cells are E. coli bacteria. In some embodiments, the E. coli bacteria are E. coli CFT073.

In some embodiments, the parent cells comprise one or more heterologous nucleotide sequences encoding the components of the T1P. In some embodiments, the one or more heterologous nucleotide sequences comprise a fim operon. In some embodiments, the fim operon is the fim operon of E. coli CFT073.

In some embodiments, the one or more heterologous nucleotide sequences comprise a sequence having at least 90% identity to the nucleotide sequence of SEQ ID NO: 1. In some embodiments, the one or more heterologous nucleotide sequences comprise a sequence having at least 95% identity to the nucleotide sequence of SEQ ID NO: 1. In some embodiments, the one or more heterologous nucleotide sequences comprise the nucleotide sequence of SEQ ID NO: 1.

In some embodiments, the one or more heterologous nucleotide sequences further comprise a constitutive promoter operably linked to the fim operon.

In some embodiments, the constitutive promoter is a modified fimS promoter comprising a mutation at a recombinase cleavage site that prevents recombination of the fimS promoter into an ‘OFF’ orientation.

In some embodiments, the one or more nucleotide sequences encoding the components of the T1P are carried on a vector. In some embodiments, the parent bacterial cells have been transiently transformed with the vector. In some embodiments, the parent bacterial cells have been stably transformed with the vector.

In some embodiments, the parent cells are Gram-negative bacterial cells. In some embodiments, the Gram-negative bacterial cells are E. coli, Salmonella, Yersinia, Vibrio, Pseudomonas, Shigella, or Legionella bacterial cells.

In some embodiments, the parent cells do not comprise a complete endogenous fim operon.

In some embodiments, the parent cells have not been exposed to a culture condition that promotes the expression of the fim operon. In some embodiments, the culture condition is temperature, pH, osmolality, shaking, or activation of the stress or stringent response.

In some embodiments, the ADAS comprise a cargo.

In some embodiments, the cargo is a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP).

In some embodiments, the cargo is encapsulated by the ADAS. In some embodiments, the cargo is attached to the surface of the ADAS.

In some embodiments, the ADAS comprise a heterologous bacterial secretion system. In some embodiments, the heterologous bacterial secretion system is a type 3 secretion system (T3SS).

In some embodiments, the cargo comprises a moiety that directs export by the bacterial secretion system.

In another aspect, the disclosure features a composition comprising the plurality of ADAS of any one of the above embodiments.

In some embodiments, the composition is formulated for delivery to a mammal. In some embodiments, the composition is formulated for oral delivery.

In another aspect, the disclosure features a method for delivering an ADAS to a cell, the method comprising (a) providing a composition comprising the plurality of ADAS of any one of the above embodiments; and (b) contacting the cell with the composition of step (a).

In some embodiments, delivery of the ADAS to the cell is increased by at least 10% relative to an ADAS derived from an unmodified parent cell.

In some embodiments, an effective amount of the ADAS is delivered to the cell at a dose that is at least 10% lower than the dose required for an ADAS derived from an unmodified parent cell. In another aspect, the disclosure features a method for delivering a cargo to a cell, the method comprising (a) providing a composition comprising a plurality of the ADAS of any one of the above embodiments, wherein the ADAS further comprise a cargo; and (b) contacting the cell with the composition of step (a).

In some embodiments, the ADAS further comprise a heterologous bacterial secretion system. In some embodiments, the heterologous bacterial secretion system is a T3SS.

In some embodiments, the delivery is to the cytoplasm of the cell.

In some embodiments, delivery of the cargo to the cell is increased by at least 10% relative to an ADAS derived from an unmodified parent cell.

In some embodiments, an effective amount of the cargo is delivered to the cell at a dose that is at least 10% lower than the dose required for an ADAS derived from an unmodified parent cell.

In another aspect, the disclosure features a method of modulating a cell, the method comprising (a) providing a composition comprising the plurality of ADAS of any one of the above embodiments; and (b) contacting the cell with the composition of step (a), whereby the cell is modulated.

In some embodiments, the cell is a mammalian cell.

In some embodiments, the mammalian cell is a gut cell. In some embodiments, the gut cell is a gut-associated lymphoid tissue (GALT) cell, a Peyer's patch cell, an M cell, a lamina propria cell, a small intestine cell, or a large intestine cell.

In some embodiments, the mammalian cell is a bladder cell.

In some embodiments, the mammalian cell is an immune cell.

In some embodiments, the mammalian cell is a blood-brain barrier cell.

In some embodiments, the mammalian cell is a mannosylated cell.

In another aspect, the disclosure features a method of treating a mammal in need thereof, the method comprising (a) providing a composition comprising the plurality of ADAS of any one of the above embodiments; and (b) contacting the mammal with an effective amount of the composition of step (a), thereby treating the mammal.

In some embodiments, a therapeutic effect is achieved at a dose that is at least 10% lower than the dose required for an ADAS derived from an unmodified parent cell.

In another aspect, the disclosure features an ADAS comprising a T1P derived from a parent bacterial cell, wherein the ADAS is produced by a process comprising the steps of (a) providing a parent cell that has been modified to constitutively express the components of a T1P; and (b) producing an ADAS from the parent bacterial cell, wherein the ADAS comprises the T1P.

In another aspect, the disclosure features an ADAS comprising a T1P derived from a parent bacterial cell, wherein the ADAS is produced by a process comprising the steps of (a) providing a parent cell that has been modified to express the components of a native T1P at a level that is at least 1.5-fold greater than a level observed in an unmodified parent cell; and (b) producing an ADAS from the parent bacterial cell, wherein the ADAS comprises the T1P that is native to the parent cell.

In another aspect, the disclosure features an ADAS comprising a T1P derived from a parent bacterial cell, wherein the ADAS is produced by a process comprising the steps of (a) providing a parent cell that has been modified to express the components of a heterologous T1P; and (b) producing an ADAS from the parent bacterial cell, wherein the ADAS comprises the T1P that is heterologous to the parent cell.

In another aspect, the disclosure features an engineered bacterium constitutively expressing the components of a T1P, wherein the engineered bacterium comprises the T1P at a level which is at least 1.5-fold greater compared to the level of T1P comprised by a non-engineered bacterium.

In some embodiments, the T1P is a native T1P. In other aspects, the TIP is a heterologous T1P.

In another aspect, the disclosure features an engineered bacterium that constitutively expresses the components of a T1P, wherein the bacterium has been modified to produce ADAS.

In another aspect, the disclosure features a method for producing an ADAS, the method comprising the steps of (a) providing an engineered bacterium that constitutively expresses the components of a T1P; and (b) producing an ADAS from the bacterium.

In another aspect, the disclosure features an ADAS produced according to any one of the above-described methods.

Other features and advantages of the invention will be apparent from the following Detailed Description and the Claims.

### BRIEF DESCRIPTION OF THE DRAWINGS

FIG. 1 is a schematic diagram showing two conformations of the fim operon, which comprises the genes encoding the type 1 pilus (T1P). The promoter (fimS) is phase-variable, and is flanked by inverted repeat (IR) sites (shown as boxes) that can be cleaved by the site-specific recombinases fimB and fimE. Cleavage and inversion of the promoter flip the promoter between an ‘ON’ orientation (promoter faces the operon; fim operon is expressed) and an ‘OFF’ orientation (promoter faces away from the operon, no expression of fim operon). The recombinase cleavage site targeted by Lambda-RED recombineering to produce a locked-on (LON) version of the operon is shown in green.

FIG. 2 is a set of electron micrographs showing achromosomal dynamic active systems (ADAS) derived from control K12 E. coli (strain BW25113) and pathogenic E. coli (strain CFT073) parent cells or from parent cells that have been engineered to comprise a locked-on (LON) version of the fim operon (LON T1P).

FIG. 3 is a set of bar graphs showing the hemagglutination titer (2x dilution series) of control parent bacterial cells (E. coli strains BW25113 and CFT073), parent bacterial cells that have been engineered to comprise a LON version of the fim operon (LON T1P), and ADAS produced therefrom. Parent cells or ADAS were serially diluted in PBS and were contacted with guinea pig RBCs. PBS comprising exogenous mannose was included as a functional control.

FIG. 4 is a bar graph showing the number of ADAS counted per HT-29 cell nucleus, as measured in an assay for binding of ADAS produced from BW25113 or BW25113 LON T1P parent cells to the human colorectal cancer cell line HT-29. A preparation including exogenous mannose was provided as a control.

### DETAILED DESCRIPTION OF THE INVENTION

### I. Definitions

As used herein, the term “achromosomal dynamic system” or “ADAS” refers to a genome-free, non-replicating, enclosed membrane system comprising at least one membrane and having an interior volume suitable for containing a cargo (e.g., one or more of a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)). In some embodiments, ADAS are minicells or modified minicells derived from a parent bacterial cell (e.g., a gram-negative or a gram-positive bacterial cell). In other aspects, ADAS are derived from parent cells by modifying the parent cell to remove the genome, and are substantially similar in size to the parent cell. ADAS may be derived from parent bacteria using any suitable method, e.g., genetic manipulation of the parent cell or exposure to a culture or condition that increases the likelihood of formation of bacterial minicells. Exemplary methods for making ADAS are those that disrupt the cell division machinery of the parent cell. In some embodiments, ADAS may comprise one or more endogenous or heterologous features of the parent cell surface, e.g., cell walls, cell wall modifications, flagella, or pili (e.g., type 1 pili (T1P), and/or one or more endogenous or heterologous features of the interior volume of the parent cell, e.g., nucleic acids, plasmids, proteins, small molecules, transcription machinery, or translation machinery. In other embodiments, ADAS may lack one or more features of the parent cell. In still other embodiments, ADAS may be loaded or otherwise modified with a feature not comprised by the parent cell.

As used herein the term “T1P-ADAS” refers to an ADAS that comprises a type 1 pilus (T1P) at or above a detectable threshold level. T1P may be detected using, e.g., a red blood cell (RBC) hemagglutination assay, as described herein.

As used herein, the terms “constitutive expression” and “constitutively expressed”, as used in reference to a T1P, refer to a condition in which the nucleic acid sequence or sequences encoding the components of the T1P are expressed (e.g., transcribed and translated (e.g., expressed by a bacterial cell, ADAS, or other system comprising the nucleic acid sequence or sequences)) regardless of environmental conditions, i.e., a condition in which expression of the nucleic acid sequence or sequences encoding the components of the T1P cannot be turned off.

As used herein, the term “endogenous type 1 pilus or “endogenous T1P” refers to a T1P that is present on a cell (e.g., a parent cell), or an ADAS derived therefrom, and is naturally encoded by the cell (e.g., is encoded by a wild-type version of the cell). The T1P may be expressed by the endogenous genes of the cell (e.g., an endogenous fim operon), and/or may be encoded and expressed by a synthetic construct in the cell. Expression or abundance of an endogenous T1P may be increased, e.g., by the addition of a moiety that increases the abundance of the T1P (e.g., a transcriptional activator of the T1P) or reduction or removal of a negative regulator of expression of the T1P. In some embodiments, expression of the T1P is increased by modification or replacement of the native promoter of the endogenous genes encoding the T1P, e.g., modification of the fimS promoter (e.g., modification to lock the fimS promoter in an “ON” configuration.

As used herein, the term “heterologous type 1 pilus” or “heterologous T1P” refers to a T1P that is present on a cell (e.g., a parent cell), or an ADAS derived therefrom, and is not naturally encoded by the cell (e.g., is not encoded by a wild-type version of the cell). The cell may encode another T1P, or may not encode any T1P. In some embodiments, the T1P is expressed by one or more synthetic constructs in the cell.

As used herein, the term “highly active ADAS” refers to an ADAS (e.g., a T1P-ADAS) having high work potential, e.g. an ADAS having the capability to do a significant amount of useful work. Work may be metabolic work, including chemical synthesis (e.g., of proteins, nucleic acids, lipids, carbohydrates, polymers, or small molecules), chemical modification (e.g., of proteins, nucleic acids, lipids, carbohydrates, polymers or small molecules), or transport (e.g., import, export, or secretion, e.g., secretion by a bacterial secretion system (e.g., T3SS)) under suitable conditions. In certain embodiments, highly active ADAS begin with a large pool of energy, e.g., energy in the form of ATP. In other embodiments, ADAS have the capacity to take up or generate energy/ATP from another source. Highly active ADAS may be identified, e.g., by having increased ATP concentration, increased ability to generate ATP, increased ability to produce a protein, increased rate or amount of production of a protein, and/or increased responsiveness to a biological signal, e.g., induction of a promoter.

As used herein, the term “parent bacterial cell” refers to a cell (e.g., a gram-negative or a gram-positive bacterial cell) from which an ADAS (e.g., a T1P-ADAS) is derived. Parent bacterial cells are typically viable bacterial cells. The term “viable bacterial cell” refers to a bacterial cell that contains a genome and is capable of cell division. Preferred parent bacterial cells are derived from any of the strains in Table 1.

An ADAS composition or preparation that is “substantially free of” parent bacterial cells and/or viable bacterial cells is defined herein as a composition having no more than 500, e.g., 400, 300, 200, 150, 100 or fewer colony-forming units (CFU) per mL. An ADAS composition that is substantially free of parent bacterial cells or viable bacterial cells may include fewer than 50, fewer than 25, fewer than 10, fewer than 5, fewer than 1, fewer than 0.1, or fewer than 0.001 CFU/mL. including no bacterial cells.

As used herein, the term “endogenous Type 3 secretion system” or “endogenous T3SS” refers to a T3SS that is present on a cell (e.g., a parent cell), or an ADAS derived therefrom, and is naturally encoded by the cell (e.g., is encoded by a wild-type version of the cell). The T3SS may be expressed by the endogenous genes of the cell, and/or may be encoded and expressed by a synthetic construct in the cell. Expression or abundance of an endogenous T3SS may be increased, e.g., by the addition of a moiety that increases the abundance of the T3SS (e.g., a transcriptional activator of the T3SS) or reduction or removal of a negative regulator of expression of the T3SS.

As used herein, the term “heterologous Type 3 secretion system” or “heterologous T3SS” refers to a T3SS that is present on a cell (e.g., a parent cell), or an ADAS derived therefrom, and is not naturally encoded by the cell (e.g., is not encoded by a wild-type version of the cell). The cell may encode another T3SS, or may not encode any T3SS. In some embodiments, the T3SS is expressed by a synthetic construct in the cell.

As used herein, an “endogenous effector” of a secretion system (e.g., a T3SS, T4SS, or T6SS) is a moiety (e.g., a protein or polypeptide) that is naturally encoded by a cell from which the secretion system (e.g., T3SS) is derived (e.g., is encoded by a wild-type version of the cell) and is capable of being secreted by the secretion system. A secretion system and one or more of its endogenous effectors may be expressed in the cell in which they naturally occur or may be expressed heterologously, e.g., expressed by a cell that does not naturally encode the endogenous effector or the secretion system.

As used herein, an effector that is heterologous with respect to a secretion system (“heterologous effector”) is a moiety (e.g., a protein or polypeptide) that is not naturally encoded by a cell from which the secretion system (e.g., T3SS) is derived (e.g., is not encoded by a wild-type version of the cell) and is capable of being secreted by a secretion system of a cell from which the heterologous effector is derived. The effector may be capable of being secreted by the secretion system to which it is heterologous, or may be modified to be secreted by the secretion system to which it is heterologous. In some embodiments, the heterologous effector is an effector of a T4SS or a T6SS that is secreted by a T3SS.

As used herein, a “cargo” of a secretion system (e.g., T3SS) is a moiety that is capable of being secreted (e.g., delivered into the cytoplasm of a host cell or into the extracellular space) by the secretion system. A cargo may be an endogenous effector of the secretion system, a heterologous effector, or a moiety that is not naturally secreted by any secretion system. The cargo may be, e.g., a protein, or a polypeptide, e.g., an enzyme (e.g., a metabolic enzyme), a DNA-modifying agent (e.g., a component of a CRISPR system), a chromatin-remodeling agent, a gene editing agent, a nuclear targeting agent (e.g., a transcription factor), a binding agent (e.g., an antibody or an antibody fragment, e.g., a VHH molecule), an immunogenic agent (e.g., an immunostimulatory or immunosuppressive agent), or a toxin. The cargo may be modified to be secreted by the secretion system, e.g., by the addition of a secretion signal (e.g., an N-terminal secretion signal), e.g., a secretion signal provided in Table 3.

As used herein, the term “percent identity” refers to percent (%) sequence identity with respect to a reference polynucleotide or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)
where X is the number of nucleotides or amino acids scored as identical matches by a sequence alignment program (e.g., BLAST) in that program's alignment of A and B, and where Y is the total number of nucleotides or amino acids in B. In some embodiments, sequence identity, for example, in homologues of MinE or DivIVA proteins will have at least about 40%, 50%, 60%, 70%, 80%, 85%, 90%, or even 95% or greater amino acid or nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% or greater amino acid sequence or nucleic acid identity, to a native sequence MinE (or minE) or DivIVA (or divIVA) sequence as disclosed herein.

The phrases “modulating a state of a cell” as used herein, refers to an observable change in the state (e.g., the transcriptome, proteome, epigenome, biological effect, or health or disease state) of the cell (e.g., a mammalian cell) as measured using techniques and methods known in the art for such a measurement, e.g., methods to measure the level or expression of a protein, a transcript, an epigenetic mark, or to measure the increase or reduction of activity of a biological pathway. Modulating the state of the cell may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In some embodiments, modulating the state of the cell involves increasing a parameter (e.g., the level or expression of a protein, a transcript, or activity of a biological pathway) of the cell. Increasing the state of the cell may result in an increase of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In other embodiments, modulating the state of involves decreasing a parameter (e.g., the level or expression of a protein, a transcript, or activity of a biological pathway) of the cell. Decreasing the state of the cell may result in a decrease of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration).

As used herein, the term “heterologous” means not native to a cell or composition in its naturally-occurring state. In some embodiments “heterologous” refers to a molecule; for example, a T1P, a component thereof, or an nucleic acid encoding the same or a cargo or payload (e.g., a polypeptide, a nucleic acid such as a protein-encoding RNA or tRNA, or small molecules) or a structure (e.g., a plasmid or a gene-editing system) that is not found naturally in an ADAS or the parent bacteria from which it is produced (e.g., a gram-negative or gram-positive bacterial cell).

### II. Compositions

### A. ADAS Comprising a Type 1 Pilus

An “ADAS” is a genome-free, non-replicating, enclosed membrane system comprising at least one membrane (in some embodiments, two membranes, where the two membranes are non-intersecting) and having an interior volume suitable for containing a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)). In some embodiments, ADAS are minicells or modified minicells derived from a parent bacterial cell (e.g., a gram-negative or a gram-positive bacterial cell). ADAS may be derived from parent bacteria using any suitable method, e.g., genetic manipulation of the parent cell or exposure to a culture or condition that increases the likelihood of formation of bacterial minicells. Exemplary methods for the production of ADAS are provided in WO 2020/123569.

The invention is based, at least in part, on Applicant's development of ADAS comprising type 1 pili (T1P-ADAS). T1P are hair-like structures found on the surface of bacteria. They have been shown to promote tropism to areas within the intestines (e.g., Peyer's patches) and other body sites and cell types. ADAS comprising T1P on their surface may be derived from parent bacteria comprising T1P on their surface. However, expression of the genes encoding the components of the T1P (the fim operon) is controlled by a phase-variable promoter, and production of T1P may be turned off in response to growth conditions (Zhang et al., Proc Natl Acad Sci USA, 113 (15): 4182-4187, 2016), thus making consistent production of T1P-ADAS from parent cells difficult. Further, not all desired parent bacteria possess a functional copy of the fim operon. The present invention provides ADAS derived from parent bacterial cells that constitutively express the components of a T1P and/or comprise one or more heterologous nucleotide sequences encoding the components of a T1P; methods of producing the same; and methods of delivering such ADAS and/or cargoes thereof to cells.

In some embodiments, an ADAS has a major axis cross section between about 100 nm-500 μm (e.g., in certain embodiments, about: 100-600 nm, such as 100-400 nm; or between about 0.5-10 μm, and 10-500 μm). In certain embodiments, an ADAS has a minor axis cross section between about: 0.001, 0.01, 0.1, 1, 2, 3, 4, 5, 6, 7, 8, 9, 10, 11, 12, 13, 14, 15, 20, 30, 40, 50, 60, 70, 80, 90, up to 100% of the major axis. In certain embodiments, an ADAS has an interior volume of between about: 0.001-1 μm³, 0.3-5 μm³, 5-4000 μm³, or 4000-50×10⁷ μm³. In some embodiments, the ADAS is substantially similar in size to the parent cell, e.g., has a size (e.g., interior volume, major axis cross-section, and/or minor axis cross section) that is about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the size of the parent cell, has a size that is identical to that of the parent cell, or has a size that is about 101%, 102%, 103%, 104%, 105%, 106%, 107%, 108%, 109%, or 110% of the size of the parent cell.

ADAS can be derived from different sources including a parental bacterial strain (“parental strain”) engineered or induced to produce genome-free enclosed membrane systems; a genome-excised bacterium; a bacterial cell preparation extract (e.g., by mechanical or other means); or a total synthesis, optionally including fractions of a bacterial cell preparation.

i. ADAS Derived from Parent Cells that Constitutively Express T1P Components

In some aspects, the disclosure features a plurality of achromosomal dynamic active systems comprising a type 1 pilus (T1P-ADAS), wherein the ADAS are derived from parent bacterial cells that constitutively express the components of the T1P. In some embodiments, the components of the T1P comprise FimA, Fiml, FimC, FimD, FimF, FimG, and FimH genes.

In some embodiments, the parent cells comprise a modified fimS promoter that is operably linked to, and directs constitutive expression of, the components of the T1P. In some embodiments, the components of the T1P are encoded by a fim operon. In some aspects, the fim operon comprises genes encoding FimA, Fiml, FimC, FimD, FimF, FimG, and FimH.

The fimS promoter is flanked by two recombination sites, and cleavage and recombination at these sites allows the promoter to switch between “ON” and “OFF” configurations. In some embodiments, the parent cells comprise a modified fimS promoter operably linked to the fim operon, wherein the modified fimS promoter comprises a mutation at a recombinase cleavage site that prevents recombination of the fimS promoter into an ‘OFF’ orientation. In some embodiments, the recombination site comprising the mutation is upstream of the fimS promoter. In other embodiments, the recombination site comprising the mutation is downstream of the fimS promoter. In still other embodiments, both the upstream and downstream recombination sites are mutated. Exemplary mutations in the upstream (left) recombination site are shown in SEQ ID NOs: 4-7. SEQ ID NO: 4 shows the wild-type sequence of the fimS site of the E. coli strain CFT073 in the ‘ON’ orientation; SEQ ID NO: 5 shows a version of this fimS site comprising mutations in the left inverted repeat site that lock the promoter in the ‘ON’ orientation. SEQ ID NO: 6 shows the wild-type sequence of the fimS site of the E. coli strain MG1655 in the ‘ON’ orientation; SEQ ID NO: 7 shows a version of this fimS site comprising mutations in the left inverted repeat site that lock the promoter in the ‘ON’ orientation. In other aspects, the parent cells comprise a constitutive promoter (e.g., a J23100, J23101, J23102, J23103, J23104, J23105, J23106, J23107, J23108, J23109, J23110, J23111, J23112, J23113, J23114, J23115, J23116, J23117, J23118, J23119, J23150, or J23151 promoter) that is operably linked to, and directs constitutive expression of, the components of the T1P (e.g., the fim operon). Exemplary promoter sequences are provided, e.g., at the GEM Registry of Standard Biological Parts. In some aspects, the endogenous fimS promoter has been replaced by the constitutive promoter.

In other aspects, the disclosure features a plurality of T1P-ADAS, wherein the ADAS are derived from parent bacterial cells that can be induced to express the components of the T1P. In some aspects, the parent cells comprise an inducible promoter (e.g., a pTac, pTrp, Para, RLac, pTet, or pRha promoter) that is operably linked to, and directs inducible expression of, the components of the T1P (e.g., the fim operon). In some aspects, the endogenous fimS promoter has been replaced by the inducible promoter. In some embodiments, the parent cells are E. coli bacteria. In some embodiments, the E. coli bacteria are E. coli CFT073.

In some embodiments, the parent cells express the components of the T1P at a level that is at least 1.1-fold greater than a level observed in an unmodified parent cell (e.g., at least 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or 100-fold or more than 100-fold greater than a level observed in an unmodified parent cell, e.g., 1-fold to 2-fold, 2-fold to 5-fold, 5-fold to 10-fold, 10-fold to 20-fold, 20-fold to 30-fold, 30-fold to 40-fold, 40-fold to 50-fold, 50-fold to 100-fold, or more than 100-fold greater than a level observed in an unmodified parent cell). In some embodiments, the parent cells express the components of the T1P at a level that is at least 1.5-fold greater than a level observed in an unmodified parent cell.

In some embodiments, the ADAS comprise the T1P at a level that is that is at least 1.1-fold greater than a level observed in a plurality of ADAS produced from control (e.g., unmodified) parent cells (e.g., at least 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or 100-fold or more than 100-fold greater than a level observed in a plurality of ADAS produced from control parent cells, e.g., 1-fold to 2-fold, 2-fold to 5-fold, 5-fold to 10-fold, 10-fold to 20-fold, 20-fold to 30-fold, 30-fold to 40-fold, 40-fold to 50-fold, 50-fold to 100-fold, or more than 100-fold greater than a level observed in a plurality of ADAS produced from control parent cells). In some embodiments, the ADAS comprise the T1P at a level that is at least 1.5-fold greater than a level observed in a plurality of ADAS produced from control parent cells.

In some embodiments, the proportion of the plurality of ADAS comprising a T1P is increased relative to a plurality of ADAS produced by parent cells that do not constitutively express the components of a T1P, e.g., increased by at least 1%, 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., increased by 1%-5%, 5%-10%, 10%-20%, 20%-30%, 30%-40%, 40%-50%, 50%-60%, 60%-70%, 70%-80%, 80%-90%, 90%-100%, or more than 100%.

In some aspects, the disclosure provides an ADAS comprising a T1P derived from a parent bacterial cell, wherein the ADAS is produced by a process comprising the steps of (a) providing a parent cell that has been modified to express the components of a native T1P at a level that is at least 1.5-fold greater than a level observed in an unmodified parent cell (e.g., (e.g., at least 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or 100-fold or more than 100-fold greater than a level observed in an unmodified parent cell, e.g., 1-fold to 2-fold, 2-fold to 5-fold, 5-fold to 10-fold, 10-fold to 20-fold, 20-fold to 30-fold, 30-fold to 40-fold, 40-fold to 50-fold, 50-fold to 100-fold greater than a level observed in an unmodified parent cell); and (b) producing an ADAS from the parent bacterial cell, wherein the ADAS comprises the T1P that is native to the parent cell. In some embodiments, the comparison between the T1P-ADAS and the unmodified parent cell is made under conditions in which an unmodified parent cell does not produce T1P or produces T1P at a low level. In unmodified bacterial cells, the native promoter for the type 1 pilus (T1P), fimS, is phase variable and may switch between ‘ON’ and ‘OFF’ orientations in response to numerous signals, including temperature, pH, osmolality, and the stress and stringent responses. In the lab, growing E. coli in LB broth at 37° C., shaking (the standard growth condition for E. coli) promotes maintenance of the fimS switch in the ‘OFF’ orientation. Switching growth to 37° C., static (rather than shaking) promotes maintenance of the fimS switch in the ‘ON’ orientation. Thus, in some aspects, the disclosure provides an ADS comprising a T1P derived from a parent bacterial cell which has not been exposed to a culture condition that promotes the expression of the fim operon (e.g., has not been exposed to such a condition prior to the production of ADAS or has not been exposed to such a condition during the generation of ADAS), wherein the ADAS is produced by a process comprising the steps of (a) providing a parent cell that has been modified to express the components of a native T1P at a level that is at least 1.5-fold greater than a level observed in an unmodified parent cell (e.g., (e.g., at least 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or 100-fold or more than 100-fold greater than a level observed in an unmodified parent cell, e.g., 1-fold to 2-fold, 2-fold to 5-fold, 5-fold to 10-fold, 10-fold to 20-fold, 20-fold to 30-fold, 30-fold to 40-fold, 40-fold to 50-fold, 50-fold to 100-fold greater than a level observed in an unmodified parent cell); and (b) producing an ADAS from the parent bacterial cell, wherein the ADAS comprises the T1P that is native to the parent cell.

In some embodiments, the culture condition is temperature, pH, osmolality, shaking, or activation of the stress or stringent response. In some embodiments, the culture condition that promotes the expression of the fim operon is growth at 37° C. under static (rather than shaking) conditions. Conditions affecting the expression of the fim operon are further described in Zhang et al., Proc Natl Acad Sci USA, 113 (15): 4182-4187, 2016.

ii. ADAS Derived from Parent Cells Comprising Heterologous T1P Components

In some aspects, the plurality of ADAS described above are derived from parent cells that comprise one or more heterologous nucleotide sequences encoding the components of the T1P. In some embodiments, the one or more heterologous nucleotide sequences comprise a fim operon. In some embodiments, the fim operon is the fim operon of E. coli CFT073.

In some embodiments, the one or more heterologous nucleotide sequences comprise a sequence having at least 90% identity to the nucleotide sequence of SEQ ID NO: 1, an E. coli CFT073 fim operon sequence (e.g., comprise a sequence having at least 91%, at least 92%, at least 93%, at least 94%, at least 95%, at least 96%, at least 97%, at least 98%, at least 99%, or more than 99% identity to the nucleotide sequence of SEQ ID NO: 1, e.g., 90%-92%, 92%-94%, 94%-96%, 96%-98%, or 98%-100% identity). In some embodiments, the one or more heterologous nucleotide sequences comprise a sequence having at least 95% identity to the nucleotide sequence of SEQ ID NO: 1. In some embodiments, the one or more heterologous nucleotide sequences comprise the nucleotide sequence of SEQ ID NO: 1.

In some embodiments, the one or more heterologous nucleotide sequences comprise a sequence having at least 90% identity to the nucleotide sequence of SEQ ID NO: 3, an E. coli MG1655 fim operon sequence (e.g., comprise a sequence having at least 91%, at least 92%, at least 93%, at least 94%, at least 95%, at least 96%, at least 97%, at least 98%, at least 99%, or more than 99% identity to the nucleotide sequence of SEQ ID NO: 3, e.g., 90%-92%, 92%-94%, 94%-96%, 96%-98%, or 98%-100% identity). In some embodiments, the one or more heterologous nucleotide sequences comprise a sequence having at least 95% identity to the nucleotide sequence of SEQ ID NO: 3. In some embodiments, the one or more heterologous nucleotide sequences comprise the nucleotide sequence of SEQ ID NO: 3.

In some embodiments, the one or more heterologous nucleotide sequences are operably linked to a constitutive promoter. In some embodiments, the one or more heterologous nucleotide sequences further comprise a constitutive promoter operably linked to the fim operon. In some embodiments, the constitutive promoter is a modified fimS promoter comprising a mutation at a recombinase cleavage site that prevents recombination of the fimS promoter into an ‘OFF’ orientation (e.g., comprising a mutation at a restriction site upstream of the fimS promoter; comprising a mutation at a restriction site downstream of the fimS promoter; or comprising mutations at both the upstream and downstream recombination sites).

In some embodiments, the one or more nucleotide sequences encoding the components of the T1P are carried on a vector. In some embodiments, the parent bacterial cells have been transiently transformed with the vector. In other embodiments, the parent bacterial cells have been stably transformed with the vector.

In some embodiments, the parent cells comprise a functional endogenous fim operon.

In some embodiments in which the parent cells comprise a functional endogenous fim operon, the parent cells express the components of the T1P at a level that is at least 1.1-fold greater than a level observed in an unmodified parent cell (e.g., at least 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or 100-fold or more than 100-fold greater than a level observed in an unmodified parent cell, e.g., 1-fold to 2-fold, 2-fold to 5-fold, 5-fold to 10-fold, 10-fold to 20-fold, 20-fold to 30-fold, 30-fold to 40-fold, 40-fold to 50-fold, 50-fold to 100-fold, or more than 100-fold greater than a level observed in an unmodified parent cell). In some embodiments, the parent cells express the components of the T1P at a level that is at least 1.5-fold greater than a level observed in an unmodified parent cell.

In some embodiments in which the parent cells comprise a functional endogenous fim operon, the ADAS comprise the T1P at a level that is that is at least 1.1-fold greater than a level observed in a plurality of ADAS produced from control (e.g., unmodified) parent cells (e.g., at least 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or 100-fold or more than 100-fold greater than a level observed in a plurality of ADAS produced from control parent cells, e.g., 1-fold to 2-fold, 2-fold to 5-fold, 5-fold to 10-fold, 10-fold to 20-fold, 20-fold to 30-fold, 30-fold to 40-fold, 40-fold to 50-fold, 50-fold to 100-fold, or more than 100-fold greater than a level observed in a plurality of ADAS produced from control parent cells). In some embodiments, the ADAS comprise the T1P at a level that is at least 1.5-fold greater than a level observed in a plurality of ADAS produced from control parent cells.

In some embodiments in which the parent cells comprise a functional endogenous fim operon, the proportion of the plurality of ADAS comprising a T1P is increased relative to a plurality of ADAS produced by parent cells that do not constitutively express the components of a T1P, e.g., increased by at least 1%, 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., increased by 1%-5%, 5%-10%, 10%-20%, 20%-30%, 30%-40%, 40%-50%, 50%-60%, 60%-70%, 70%-80%, 80%-90%, 90%-100%, or more than 100%.

In some embodiments, the parent cells do not comprise a complete endogenous fim operon.

In some aspects, the disclosure provides an ADAS comprising a T1P derived from a parent bacterial cell, wherein the ADAS is produced by a process comprising the steps of (a) providing a parent cell that has been modified to constitutively express the components of a T1P; and (b) producing an ADAS from the parent bacterial cell, wherein the ADAS comprises the T1P.

In some aspects, the disclosure features an ADAS comprising a T1P derived from a parent bacterial cell, wherein the ADAS is produced by a process comprising the steps of (a) providing a parent cell that has been modified to express the components of a heterologous T1P; and (b) producing an ADAS from the parent bacterial cell, wherein the ADAS comprises the T1P that is heterologous to the parent cell.

iii. Parent Cells

In some embodiments of the T1P-ADAS of Section IIA (i) or IIA (ii), the parent cells are Gram-negative bacterial cells. Parent bacteria include any suitable bacterial species from which an ADAS may be generated (e.g., species that may be modified to produce ADAS). Table 1 provides a non-limiting list of suitable Gram-negative genera from which ADAS can be derived.

TABLE 1
Bacterial genera for ADAS production
Gram negative bacteria

| | | | |
|---|---|---|---|
| Escherichia | Chromobacterium | Methylococcus | Shewanella |
| Acinetobacter | Crocosphaera | Neisseria | Shigella |
| Agrobacterium | Coxiella | Nitrosomonas | Sinorhizobium |
| Anabaena | Dechloromonas | Nostoc | Synechococcus |
| Anaplasma | Desulfitobacterium | Photobacterium | Synechocystis |
| Aquifex | Desulfotalea | Photorhabdus | Thermosynechococcus |
| Azoarcus | Erwinia | Phyllobacterium | Thermotoga |
| Azotobacter | Francisella | Polaromonas | Thermus |
| Azospirillum | Fusobacterium | Prochlorococcus | Thiobacillus |
| Bartonella | Gloeobacter | Pseudomonas | Trichodesmium |
| Bordetella | Gluconobacter | Psychrobacter | Vibrio |
| Bradyrhizobium | Helicobacter | Ralstonia | Wigglesworthia |
| Brucella | Legionella | Rhizobium | Wolinella |
| Buchnera | Magnetospirillum | Rickettsia | Xanthomonas |
| Burkholderia | Mesorhizobium | Rubrivivax | Xylella |
| Candidatus | Methylobacterium | Salmonella | Yersinia |

In some embodiments, the Gram-negative bacterial cells are Escherichia (e.g., E. coli), Salmonella, Yersinia, Vibrio, Pseudomonas, Shigella, or Legionella bacterial cells. In some embodiments, the parent bacterial cell is a probiotic cell. In some embodiments, the parent bacterial cell is a mammalian pathogen or a mammalian commensal bacterium. In some instances, the mammalian commensal bacterium is a Staphylococcus, Bifidobacterium, Micrococcus, Lactobacillus, or Actinomyces species or the mammalian pathogenic bacterium is enterohemorrhagic Escherichia coli (EHEC), Salmonella typhimurium, Shigella flexneri, Yersinia enterolitica, or Helicobacter pylori.

In some embodiments, the parent bacterial cell is an auxotrophic parent bacterium, i.e., a parent bacterium that is unable to synthesize an organic compound required for growth. Such bacteria are able to grow only when the organic compound is provided.

In some embodiments, the parent cells are E. coli bacteria. In some embodiments, the E. coli bacteria are E. coli CFT073.

In some embodiments, the parent cell has not been exposed to a culture condition that promotes the expression of the fim operon, e.g., has not been exposed to such a condition prior to the production of ADAS or has not been exposed to such a condition during the generation of ADAS. In some embodiments, the culture condition is temperature, pH, osmolality, shaking, or activation of the stress or stringent response. In some embodiments, the culture condition that promotes the expression of the fim operon is growth at 37° C. under static (rather than shaking) conditions. Conditions affecting the expression of the fim operon are further described in Zhang et al., Proc Natl Acad Sci USA, 113 (15): 4182-4187, 2016.

In some aspects, the disclosure features an engineered bacterium constitutively expressing the components of a T1P, wherein the engineered bacterium comprises the T1P at a level which is at least 1.1-fold greater compared to the level of T1P comprised by a non-engineered bacterium (e.g., at least 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or 100-fold or more than 100-fold greater than a level observed in an unmodified parent cell, e.g., 1-fold to 2-fold, 2-fold to 5-fold, 5-fold to 10-fold, 10-fold to 20-fold, 20-fold to 30-fold, 30-fold to 40-fold, 40-fold to 50-fold, 50-fold to 100-fold, or more than 100-fold greater compared to the level of T1P comprised by a non-engineered bacterium). In some embodiments, the engineered bacterium comprises the T1P at a level which is at least 1.5-fold greater compared to the level of T1P comprised by a non-engineered bacterium. In some embodiments, the T1P is a native T1P, e.g., a T1P encoded by a fim operon that is endogenous to the bacterium. In some embodiments, the T1P is a heterologous T1P, e.g., a T1P encoded by one or more nucleotide sequences (e.g., a fim operon) that are heterologous to the bacterium. In some embodiments, the bacterium has been modified to produce ADAS.

In some aspects, the disclosure features an engineered bacterium that constitutively expresses the components of a T1P, wherein the bacterium has been modified to produce ADAS.

In some aspects, the disclosure features an ADAS produced by a method comprising the steps of (a) providing an engineered bacterium that constitutively expresses the components of a T1P; and (b) producing an ADAS from the bacterium.

### B. ADAS Comprising a Cargo

In some embodiments, an ADAS provided by the invention (e.g., a T1P-ADAS) includes a cargo, e.g., a cargo contained in the interior of the ADAS. A cargo may be any moiety disposed in the interior of an ADAS (e.g., encapsulated by the ADAS) or conjugated to the surface of the ADAS. In some embodiments, the cargo comprises a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP) or a combination of the foregoing. In some aspects, the cargo is delivered by a secretion system (e.g., T3SS) comprised by the ADAS. In other aspects, the cargo is not delivered by a T3SS.

In some embodiments, the nucleic acid is a DNA, an RNA, or a plasmid. In some embodiments, the nucleic acid (e.g., DNA, RNA (e.g., mRNA, ASO, circular RNA (circRNA), siRNA, shRNA, tRNA, dsRNA, or a combination thereof), or plasmid) encodes a protein. In some embodiments, the protein is transcribed and/or translated in the ADAS. In some embodiments, the nucleic acid inhibits translation of a protein or polypeptide, e.g., is an siRNA or an antisense oligonucleotide (ASO).

In some embodiments, the cargo is an agent that can modulate the microbiome of the target organism (e.g., a human microbiome or a non-human mammalian microbiome), e.g., a polysaccharide, an amino acid, an anti-microbial agent (e.g., e.g., an anti-infective or antimicrobial peptide, protein, and/or natural product), a short chain fatty acid, or a combination thereof. In some examples, the agent that can modulate the host microbiome is a probiotic agent.

In some embodiments, the cargo is an enzyme. In some embodiments, the enzyme alters a substrate to produce a target product. In some embodiments, the substrate is present in the ADAS and the target product is produced in the ADAS. In other embodiments, the substrate is present in a target cell or environment to which the ADAS is delivered.

In certain embodiments, the cargo is modified for improved stability compared to an unmodified version of the cargo. “Stability” of a cargo is a unitless ratio of half-life of unmodified cargo and modified cargo half-life, as measured in the same environmental conditions. In some embodiments, the environment is experimentally controlled, e.g., a simulated body fluid, RNAse free water, cell cytoplasm, extracellular space, or “ADAS plasm” (i.e., the content of the interior volume of an ADAS, e.g., after lysis). In other embodiments, the environment is an actual or simulated: mammalian gut, mammalian skin, mammalian reproductive tract, mammalian respiratory tract, mammalian blood stream, or mammalian extracellular space. In certain embodiments, the ADAS does not substantially degrade the cargo.

In certain embodiments, the cargo comprises a protein. In certain embodiments, the protein has stability greater than about 1.01, 1.1, 10, 100, 1000, 10000, 100000, 100000, or 10000000 in cell cytoplasm or other environments. The protein can be any protein, including growth factors; enzymes; hormones; immune-modulatory proteins; antibiotic proteins, such as antibacterial, antifungal, or antiviral proteins, etc.; and targeting agents, such as antibodies or nanobodies, etc. In some embodiments, the protein is a hormone, e.g., a paracrine, endocrine, or autocrine hormone.

In some embodiments, the cargo is an anti-inflammatory agent, e.g., a cytokine (e.g., a heterologously expressed anti-inflammatory cytokine or mutein thereof (e.g., IL-10, TGF-Beta, IL-22, IL-2) or antibody (e.g., an antibody or antibody fragment targeting tumor necrosis factor (TNF) (e.g., an anti-TNF antibody); an antibody or antibody fragment targeting IL-12 (e.g., an anti-IL-12 antibody); or an antibody or antibody fragment targeting IL-23 (e.g., an anti-IL-23 antibody).

In certain embodiments, the cargo is an immune modulator. Immune modulators include, e.g., immune stimulators; checkpoint inhibitors (e.g., inhibitors of PD-1, PD-L1, or CTLA-4); chemotherapeutic agents; immune suppressors; antigens; super antigens; and small molecules (e.g., cyclosporine A, cyclic dinucleotides (CDNs), or STING agonists (e.g., MK-1454)). In some embodiments, the immune modulator is a moiety that induces tolerance in a subject, e.g., an allergen, a self-antigen (e.g., a disease-associated self-antigen), or a microbe-specific antigen. In some embodiments, the immune modulator is a vaccine, e.g., an antigen from a pathogen (e.g., a virus (e.g., a viral envelope protein) or a bacteria). In some embodiments, the antigen is a cancer neo-antigen. In some embodiments, the pathogen is a coronavirus, e.g., SARS-COV-2. In some embodiments, the cargo an adjuvant, e.g., an immunomodulatory molecule or a molecule that alters the compartmentalization, presentation, or profile of one or more co-stimulatory molecules associated with a vaccine antigen. In some examples, the adjuvant is an activator of an immune pathway upstream of a desired immune response (e.g., an activator of an innate immune pathway upstream of an adaptive immune response). In other examples, the adjuvant enhances the presentation of an antigen on an immune cell or immune moiety (e.g., MHC class 1) in the target organism. In some examples, the adjuvant is listeriolysin O (LLO). In some embodiments, an ADAS comprises an antigen and one or more adjuvants.

In some embodiments, the cargo is an agent for treatment or prevention of a cancer, e.g., an agent that decreases the likelihood that a patient will develop a cancer or an agent that treats a cancer (e.g., an agent that increases progression-free survival and/or overall survival in an individual having a cancer).

Agents for the prevention of cancer include, but are not limited to anti-inflammatory agents and growth inhibitors. Agents for the treatment of cancer (e.g., a solid tumor cancer) include, but are not limited to anti-inflammatory agents, growth inhibitors, chemotherapy agents, immunotherapy agents, anti-cancer antibodies or antibody fragments (e.g., antibodies or antibody fragments targeting cancer antigens (e.g., cancer neo-antigens)), cancer vaccines (e.g., vaccines comprising a cancer neo-antigen), agents that induce autophagy (e.g., activators such as listeria-lysin-o), cytotoxins, inflammasome inhibiting agents, immune checkpoint inhibitors (e.g., inhibitors of PD-1, PD-L1, or CTLA-4), transcription factor inhibitors, and agents that disrupt the cytoskeleton.

In some embodiments, the cargo is an enzyme. The enzyme may be an enzyme that performs a catalytic activity in a target cell or organism (e.g., in a human or a non-human mammal). In some embodiments, the catalytic activity is extracellular matrix (ECM) digestion (e.g., the enzyme is hyaluronidase and the catalytic activity is ECM digestion) or removal of toxins. In some embodiments, the enzyme is an enzyme replacement therapy, e.g., is phenylalanine hydroxylase. In some embodiments, the enzyme is a UDP-glucuronosyltransferase. In some embodiments, the enzyme has hepatic enzymatic activity (e.g., porphobilinogen deaminase (PBGD), e.g., human PBGD (hPBGD)). In some embodiments, the enzyme is a protease, oxidoreductase, or a combination thereof.

In some embodiments, the enzyme alters a substrate to produce a target product. In some embodiments, the substrate is present in the ADAS and the target product is produced in the ADAS. In other embodiments, the substrate is present in a target cell or environment to which the ADAS is delivered. In some embodiments, the enzyme is diadenylate cyclase A, the substrate is ATP, and the target product is cyclic-di-AMP.

In some embodiments, the enzyme is chemically conjugated to the ADAS membrane, optionally via a linker to the exterior membrane.

Alternatively, the cargo may be a nucleic acid that encodes any of the enzymes described herein.

In some embodiments, the cargo is an agent that activates or inhibits autophagic processes (e.g. an activator such as listeria-lysin-o or an inhibitor such as IcsB).

In some embodiments, the cargo is an anti-infective agent, e.g., an anti-microbial agent, e.g., an anti-infective or antimicrobial peptide, protein, and/or natural product.

In some embodiments, the cargo is a protein that modulates host transcriptional response e.g., a transcription factor; a protein that promotes host cell growth, e.g., a growth factor; or a protein that inhibits protein function, e.g., a nanobody. In some embodiments, the transcription factor is a human transcription factor.

For ADAS comprising cargo, in some embodiments, the cargo is an RNA, such as circular RNA, mRNA, siRNA, shRNA, ASO, tRNA, dsRNA, or a combination thereof. In certain embodiments, the RNA has stability greater than about: 1.01, 1.1, 10, 100, 1000, 10000, 100000, 100000, or 10000000, e.g., in ADAS plasm. The RNA cargo can be stabilized, in certain embodiments, e.g., with an appended step-loop structure, such as a tRNA scaffold (e.g., non-human tRNALys3 and E. coli tRNAMet (Nat. Methods, Ponchon 2007). Both have been well characterized and expressed recombinantly. However, a variety of other types could be used as well, such as aptamers, lncRNA, ribozymes, etc., RNA can also be stabilized where the ADAS is obtained from a parental strain null (or hypomorphic) for one or more ribonucleases.

In some particular embodiments, the RNA is a protein-coding mRNA. In more particular embodiments, the protein-coding mRNA encodes an enzyme (e.g., and enzyme that imparts hepatic enzymatic activity, such as human PBGD (hPBGD) mRNA) or an antigen, e.g., that elicits an immune response (such as eliciting a potent and durable neutralizing antibody titer), such as mRNA encoding CMV glycoproteins gB and/or pentameric complex (PC)). In certain particular embodiments, the RNA is a small non-coding RNA, such as shRNA, ASO, tRNA, dsRNA, or a combination thereof.

In certain embodiments, the ADAS provided by the invention includes cargo comprising at least one component of a gene editing system. Components of a “gene editing system” include (or encode) proteins (or nucleic acids encoding said proteins) that can, with suitable associated nucleic acids, modify a DNA sequence of interest, such as a genomic DNA sequence, whether e.g., by insertion or deletion of a sequence of interest, or by altering the methylation state of a sequence of interest, as well as nucleic acids associated with the function of such proteins, e.g., guide RNAs. Exemplary gene editing systems include those based on a Cas system, such as Cas9, Cpf1 or other RNA-targeted systems with their companion RNA (e.g., sequence-complementary CRISPR guide RNA), as well as Zinc finger nucleases and TAL-effectors conjugated to nucleases.

Other embodiments of ADAS provided by the invention include DNA as the cargo, including as a plasmid, optionally wherein the DNA comprises a protein-coding sequence. Exemplary DNA cargo includes, in certain embodiments, a plasmid encoding an RNA sequence of interest (see examples above), e.g., which may be flanked on each side by an tRNA insert. Various DNA cargo are encompassed by the invention, including: ADAS producing (e.g., driving FTZ overexpression, genome degrading exonucleases); longevity plasmids (ATP synthase expressing, rhodopsin-expressing); those expressing stabilized non-coding RNA, RNA, lncRNA; expressing secretion system tag proteins, NleE2 effector domain and localization tag; secretion systems T3/4SS, T5SS, T6SS; logic circuits, conditionally expressed secretion systems; and combinations thereof. In some embodiments, a logic circuit includes inducible expression or suppression cassettes, such as IPTG-inducible Plac promoter and the hrpR portion of the AND gate, and, for example, the heat-induced promoter pL (from phage lambda, which is usually suppressed by a thermolabile protein) and the hrpS portion of the AND gate. To engineer an OR gate, a system described by Rosado et al., PLOS Genetics, 2018 can be used. Briefly, a cis-repressed mRNA coding for RFP under a constitutive promoter can be used. The repression can then be removed in the presence of RAJ11 sRNA. Plasmids containing the IPTG-inducible promoter PLac and heat-induced promoter pL, both of which induce the expression of RAJ11 sRNA, can then be used. The output would then be RFP expression, which is seen in response to either input. These systems can be adapted to a variety of sensor-type functions.

ADAS provided by the invention, in some embodiments, include a transporter in the membrane. In certain embodiments, the transporter is specific for glucose, sodium, potassium, a metal ion, an anionic solute, a cationic solute, or water.

In some embodiments, the membrane of an ADAS provided by the invention comprises an enzyme. In particular embodiments, the enzyme is a protease, oxidoreductase, or a combination thereof. In some embodiments, the enzyme is chemically conjugated to the ADAS membrane, optionally via a linker to the exterior membrane.

### C. ADAS Comprising Bacterial Secretion Systems

In certain embodiments, a T1P-ADAS provided by the invention comprises a bacterial secretion system (e.g., an endogenous bacterial secretion system or a heterologous bacterial secretion system). A “bacterial secretion system” is a protein, or protein complex, that can export a cargo from the cytoplasm of a bacterial cell (or, for example, an ADAS derived therefrom) into: the extracellular space, the periplasmic space of a gram-negative bacterium, or the intracellular space of another cell. In some embodiments, the bacterial secretion system works by an active (e.g., ATP-dependent or PMF-dependent) process, and in certain embodiments the bacterial secretion system comprises a tube or a spike spanning the host cell (or ADAS) to a target cell. In other embodiments the bacterial secretion system is a transmembrane channel. Exemplary bacterial secretion systems include T3SS and T4SS (and T3/T4SS, as defined below), which are tube-containing structures where the cargo traverses through the inside of a protein tube, and T6SS, which carries the cargo at the end of a spike. Other exemplary bacterial secretion systems include T1SS, T2SS, T5SS, T7SS, Sec, and Tat, which are transmembrane.

In some embodiments, an ADAS provided by the invention (e.g. a T1P-ADAS) comprises a heterologous bacterial secretion system. In some embodiments, the heterologous bacterial secretion system is a type 3 secretion system (T3SS). In some embodiments, the cargo comprises a moiety that directs export by the bacterial secretion system.

In some embodiments, the T3SS is from a genus of Table 2.

In some embodiments, the parent bacterial cell does not comprise an endogenous T3SS.

TABLE 2
Effectors and functional classes

| | | | Modulation of | |
|---|---|---|---|---|
| | | | Inflammatory | Additional |
| Organism | Cytotoxic | Cytoskeletal remodeling | Pathways | Effectors |
| Yersinia | | YopE, YopH, YopO, YopT | YopJ, YspK, | |
| enterocolytica | | | YopM | |
| Pseudomonas | ExoU, ExoY | ExoT, ExoS | | |
| aeriginosa | | | | |
| Salmonella | | SspH2, SteC, SrfH, SpvB, | AvrA, SspH1, | SifA, SlrP, SseF, |
| enterocolitica | | SipA, SptP, SopE2 | SpvC, GogB, | SseG, PipB2, |
| | | | SseK1, SseK2, | SopD2, SSeJ, |
| | | | SseK3, GtgA, | GtgE, PipB, SifB, |
| | | | SpvD, SopE, | SteA, SteB, SteD, |
| | | | SopA, SopB | SteE, CigR, |
| | | | | SopD, SpiC |
| Shigella flexneri | | IpgB1, IpgB2, IpgD, VirA, | IpaH7.8, OspC1, | OspE2 |
| | | IcsB, IpaA | OspB, OspF, | |
| | | | OspG, IpaH9.8, | |
| | | | OspZ, IpaH4.5 | |
| Vibrio | VPA450, | VopC, VopL, VopV | VopP, VopZ, | |
| parahaemolyticus | VopT, | | VopS, VopQ | |
| | VPA1380 | | | |
| Escherichia coli | Cif | EspH, Map, Tir, EspG, | NleB, NleC, NleE, | EspF, NleG, |
| | | EspG2, EspJ, NleL, EspZ, | NleH1, NleD, | EspL, EspO |
| | | EspW, EspT, EspM | NleF, NleA | |

In some embodiments, the T3SS is a Salmonella T3SS, a Vibrio T3SS, an Escherichia T3SS, a Yersinia T3SS, a Shigella T3SS, a Pseudomonas T3SS, or a Chlamydia T3SS. In some embodiments, the Salmonella T3SS is a Salmonella enterica T3SS. In some embodiments, the Vibrio T3SS is a Vibrio parahaemolyticus T3SS. In some embodiments, the Escherichia T3SS is an enteropathogenic E. coli (EPEC) T3SS. In some embodiments, the Yersinia T3SS, is a Yersinia enterocolitica T3SS. In some embodiments, the Shigella T3SS is a Shigella flexneri T3SS.

In some embodiments, the parent bacterial cell comprises one or more heterologous nucleotide sequences encoding the components of the T3SS. In some embodiments, the one or more nucleotide sequences encoding the components of the T3SS are carried on a vector. In some embodiments, the parent bacterial cell has been transiently transformed with the vector. In some embodiments, the parent bacterial cell has been stably transformed with the vector. In some embodiments, the parent bacterial cell further comprises a moiety that increases the level of the T3SS in the ADAS. In some embodiments, the moiety is a transcriptional activator of the one or more heterologous nucleotide sequences encoding a component of theT3SS.

In another aspect, the disclosure features a T1P-ADAS derived from a parent bacterial cell, the ADAS comprising a bacterial Type 3 secretion system (T3SS) that is endogenous to the parent bacterial cell, wherein the parent bacterial cell has been modified to reduce the level of an endogenous protein or polypeptide capable of being secreted by the T3SS.

In some embodiments, the parent cell bacterial has been modified by deleting a transcriptional activator of the endogenous protein or polypeptide capable of being secreted by the T3SS.

In some embodiments, the parent bacterial cell is a Gram-negative bacterial cell.

In some embodiments, the parent bacterial cell is from a genus of Table 2.

In some embodiments, the parent bacterial cell is a Salmonella species, a Vibrio species, an Escherichia species, a Yersinia species, or a Shigella species. In some embodiments, the Salmonella species is Salmonella enterica. In some embodiments, the Vibrio species is a Vibrio parahaemolyticus.

In some embodiments, the Escherichia species is an enteropathogenic E. coli (EPEC). In some embodiments, the Yersinia species is Yersinia enterocolitica. In some embodiments, the Shigella species is Shigella flexneri.

In some embodiments, the parent bacterial cell further comprises a moiety that increases the level of the T3SS in the ADAS. In some embodiments, the moiety is a transcriptional activator of a nucleotide sequence encoding a component of the T3SS.

In some embodiments, the parent bacterial cell has been modified to reduce the level of a negative regulator of a component of the T3SS. In some embodiments, a chromosomal locus encoding the negative regulator has been deleted from the parent bacterial cell.

In some embodiments, the parent bacterial cell has been modified to reduce the level of one or more of LPS; a metabolically non-essential protein; a toxin not associated with a T3SS; an endotoxin; a flagella; and a pilus.

In some embodiments, the ADAS further comprises at least one cargo, wherein the T3SS is capable of delivering the cargo to a target cell. In some embodiments, the delivery is to the cytoplasm of the target cell.

In some embodiments, the cargo is a protein or a polypeptide.

In some embodiments, the cargo is endogenously secreted by the T3SS.

In some embodiments, the ADAS or the parent bacterial cell has been modified to increase the level of the cargo in the ADAS.

In some embodiments, the cargo is not endogenously secreted by the T3SS.

In some embodiments, the cargo is endogenously secreted by a T3SS from a species other than the ADAS T3SS species.

In some embodiments, the cargo is endogenously secreted by a Type 4 secretion system (T4SS) or a Type 6 secretion system (T6SS).

In some embodiments, the cargo has been modified for delivery by the T3SS.

In some embodiments, the cargo is an enzyme, a DNA-modifying agent, a chromatin-remodeling agent, a gene editing agent, a nuclear targeting agent, a binding agent, an immunogenic agent, or a toxin. In some embodiments, the enzyme is a metabolic enzyme. In some embodiments, the gene editing agent is a component of a CRISPR system. In some embodiments, the nuclear targeting agent is a transcription factor. In some embodiments, the binding agent is an antibody or an antibody fragment. In some embodiments, the binding agent is a VHH molecule. In some embodiments, the immunogenic agent is an immunostimulatory agent. In some embodiments, the immunogenic agent is an immunosuppressive agent.

In some embodiments, the cargo has been modified by addition of a secretion signal. In some embodiments, the secretion signal is a sequence of Table 3.

TABLE 3
Secretion signals

| | Secretion | Amino acid | |
|---|---|---|---|
| Organism | signal | length | Sequence |
| Salmonella | SptP | 180 | SEQ ID NO: 8 |
| Salmonella | SopD | 50 | SEQ ID NO: 9 |
| Salmonella | SipA | 180 | SEQ ID NO: 10 |
| Salmonella | slrP | 200 | SEQ ID NO: 11 |
| Salmonella | SopA | 100 | SEQ ID NO: 12 |
| Salmonella | SopB | 180 | SEQ ID NO: 13 |
| Salmonella | SipC | 180 | SEQ ID NO: 14 |
| Salmonella | AvrA | 100 | SEQ ID NO: 15 |
| Salmonella | OspB | 288 | SEQ ID NO: 16 |
| Salmonella | SopE2 | 120 | SEQ ID NO: 17 |
| Shigella | IpaH9.8 | 60 | SEQ ID NO: 18 |
| Shigella | OspC3 | 60 | SEQ ID NO: 19 |
| Shigella | OspG | 60 | SEQ ID NO: 20 |
| Shigella | IpaH4.5 | 60 | SEQ ID NO: 21 |
| Shigella | IpaH7.8 | 60 | SEQ ID NO: 22 |
| Pseudomonas | ExoS | 54 | SEQ ID NO: 23 |
| Yersinia | yopE | 50 | SEQ ID NO: 24 |

In some embodiments, the ADAS comprise a cargo, wherein the cargo comprises a moiety that directs export by the bacterial secretion system. In some embodiments the moiety is Pho/D, Tat, or a synthetic peptide signal.

In certain embodiments, the ADAS provided by the invention are two-membrane ADAS. In more particular embodiments, the two-membrane ADAS further comprise a bacterial secretion system. In still more particular embodiments, the bacterial secretion system is selected from T3SS, T4SS, T3/4SS, or T6SS, optionally wherein the T3SS, T4SS, T3/4SS, or T6SS have an attenuated or non-functional effector that does not affect fitness of a target cell.

In some embodiments, the bacterial secretion system is capable of exporting a cargo across the ADAS outer membrane into a target cell, such as a mammalian cell, such as T3SS, T4SS, T3/T4SS, or T6SS.

In some embodiments, the bacterial secretion system is a T3/4SS. A “T3/4SS” is a secretion system based on T3SS or T4SS, including hybrid systems as well as unmodified versions, that forms a protein tube between a bacterium (or an ADAS, e.g., a T1P-ADAS) and a target cell, connecting the two and delivering one or more effectors. In some embodiments, the target cell is a mammalian cell. In some embodiments, a T3/4SS includes an effector, which may be a modified effector. Examples of T3SS systems include the Salmonella SPI-1 system, the EHEC coli ETT1 system, the Xanthomonas citri/campestri T3SS system, and the Pseudomonas syringae T3SS system. Examples of T4SS systems include the Agrobacterium Ti plasmid system and the Helicobacter pylori T4SS. In certain embodiments, the T3/4SS has modified effector function, e.g., an effector selected from SopD2, SopE, Bop, Map, Tir, EspB, EspF, NleC, NleH2, or NleE2. In more particular embodiments, the modified effector function is for intracellular targeting such as translocation into the nucleus, golgi, mitochondria, actin, microvilli, ZO-1, microtubules, or cytoplasm. In still more particular embodiments, the modified effector function is nuclear targeting based on NleE2 derived from E. coli. In other particular embodiments, the modified effector function is for filopodia formation, tight junction disruption, microvilli effacement, or SGLT-1 inactivation.

In some embodiments, an ADAS provided by the invention comprising a bacterial secretion system comprises a T6SS. In some embodiments, the T6SS, in its natural host, targets a bacterium and contains an effector that kills the bacteria. In certain particular embodiments, the T6SS is derived from P. putida K1-T6SS and, optionally, the effector comprises the amino acid sequence of Tke2 (Accession AUZ59427.1), or a functional fragment thereof. In other embodiments, the T6SS, in its natural host, targets a fungi and contains an effector that kills fungi, e.g., the T6SS is derived from Serratia marcescens and the effectors comprise the amino acid sequences of: Tfe1 (Genbank: SMDB11_RS05530) or Tfe2 (Genbank: SMDB11_RS05390).

In some embodiments of an ADAS provided by the invention that contains a bacterial secretion system, the bacterial secretion system is capable of exporting a cargo extracellularly. In certain more particular embodiments, the bacterial secretion system is T1SS, T2SS, T5SS, T7SS, Sec, or Tat.

D. ADAS and Highly Active ADAS Derived from Parent Bacteria Deficient in a Cell Division Topological Specificity Factor

In some aspects, the invention provides a T1P-ADAS and/or a composition comprising a plurality of T1P-ADAS derived from a parent bacterium having a reduction in a level, activity, or expression of a cell division topological specificity factor.

In some aspects, the invention provides a composition comprising a plurality of T1P-ADAS, wherein the T1P-ADAS do not comprise a cell division topological specificity factor and wherein the composition is substantially free of viable bacterial cells.

In some aspects, the T1P-ADAS are produced by a process comprising: (a) making, providing, or obtaining a plurality of parent bacteria as described herein having a reduction in the level or activity of a cell division topological specificity factor; (b) exposing the parent bacterium to conditions allowing the formation of a minicell, thereby producing highly active ADAS; and (c) separating the ADAS from the parent bacterium, thereby producing a composition that is substantially free of viable bacterial cells.

In some embodiments of the above aspects, the cell division topological specificity factor is a polypeptide having an amino acid sequence with at least 20% identity to an E. coli minE polypeptide (SEQ ID NO: 25), e.g., at least 30%, 40%, 50%, 60%, 70%, 80%, 90%, 95%, 98%, or 99% identity to SEQ ID NO: 25. In some embodiments, the cell division topological specificity factor comprises the amino acid sequence of SEQ ID NO: 25. In some embodiments, the cell division topological specificity factor is a minE polypeptide. Exemplary species having minE polypeptides are provided in Table 1 and in Rothfield et al., Nature Reviews Microbiology, 3:959-968, 2005.

In some embodiments, the parent bacterium is E. coli and the minE polypeptide is E. coli minE. In other embodiments, the parent bacterium is Salmonella typhimurium and the minE polypeptide is S. typhimurium minE. In yet other embodiments, the parent bacterium is a bacterium of Table 1 and the cell division topological specificity factor is the endogenous minE or DivIVA of the parent bacterium.

In some embodiments of the above aspects, the cell division topological specificity factor is a polypeptide having an amino acid sequence with at least 20% identity to a Bacillus subtilis DivIVA polypeptide (SEQ ID NO: 28), e.g., at least 30%, 40%, 50%, 60%, 70%, 80%, 90%, 95%, 98%, or 99% identity to SEQ ID NO: 28. In some embodiments, the cell division topological specificity factor comprises the amino acid sequence of SEQ ID NO: 28. In some embodiments, the cell division topological specificity factor is a DivIVA polypeptide. Exemplary species having DivIVA polypeptides are provided in Table 1 and in Rothfield et al., Nature Reviews Microbiology, 3:959-968, 2005. In some embodiments, the parent bacterium is Bacillus subtilis and the cell division topological specificity factor is B. subtilis DivIVA.

In some embodiments, the ADAS or parent bacterium having the reduction in a level or activity of the cell division topological specificity factor also has a reduction in a level of one or more Z-ring inhibition proteins.

In some embodiments, the Z ring inhibition protein is a polypeptide having an amino acid sequence with at least 20% identity to an E. coli minC polypeptide (SEQ ID NO: 26), e.g., at least 30%, 40%, 50%, 60%, 70%, 80%, 90%, 95%, 98%, or 99% identity to SEQ ID NO: 26. In some embodiments, the Z ring inhibition protein comprises the amino acid sequence of SEQ ID NO: 26. In some embodiments, the Z ring inhibition protein is a minC polypeptide.

In some embodiments, the Z ring inhibition protein is a polypeptide having an amino acid sequence with at least 20% identity to an E. coli minD polypeptide (SEQ ID NO: 27), e.g., at least 30%, 40%, 50%, 60%, 70%, 80%, 90%, 95%, 98%, or 99% identity to SEQ ID NO: 27. In some embodiments, the Z ring inhibition protein comprises the amino acid sequence of SEQ ID NO: 27. In some embodiments, the Z ring inhibition protein is a minD polypeptide.

In some embodiments, the ADAS or parent bacterium has a reduction in the level, activity, or expression of at least two Z-ring inhibition proteins. In some embodiments, the ADAS or parent bacterium has a reduction in expression of a minC polypeptide and a minD polypeptide. In some embodiments, the ADAS or parent bacterium has a reduction in expression of a minC polypeptide, a minD polypeptide, and a minE polypeptide, e.g., a deletion of the minCDE operon (AminCDE).

A reduction in the level, activity, or expression of a cell division topological specificity factor or a Z-ring inhibition protein, e.g., a reduction in an ADAS or a reduction in a parent bacterial cell, may be achieved using any suitable method. For example, in some embodiments, the reduction in the level or activity is caused by a loss-of-function mutation, e.g., a gene deletion. In some embodiments, the loss-of-function mutation is an inducible loss-of-function mutation and loss of function is induced by exposing the parent cell to an inducing condition, e.g., the inducible loss-of-function mutation is a temperature-sensitive mutation and wherein the inducing condition is a temperature condition.

In some embodiments, the parent cell has a deletion of the minCDE operon (AminCDE) or homologous operon.

E. ADAS Lacking Proteases, RNases, and/or LPS

In another aspect, the invention provides a composition comprising a plurality of T1P-ADAS, wherein the ADAS have a reduced protease level or activity relative to an ADAS produced from a wild-type parent bacterium. In some aspects, the ADAS is produced from a parent bacterium that has been modified to reduce or eliminate expression of at least one protease.

In some embodiments, the ADAS have a reduced RNAse level or activity relative to an ADAS produced from a wild-type parent bacterium. In some embodiments, the ADAS is produced from a parent bacterium that has been modified to reduce or eliminate expression of at least one RNAse. In some embodiments, the RNase is an endoribonuclease or an exoribonuclease.

In another aspect, the invention provides a composition comprising a plurality of T1P-ADAS, wherein the ADAS has been modified to have reduced lipopolysaccharide (LPS). In some embodiments, the modification is a mutation in Lipid A biosynthesis myristoyltransferase (msbB).

In certain embodiments, a T1P-ADAS provided by the invention lacks one or more metabolically non-essential proteins. A “metabolically non-essential protein” non-exhaustively includes: fimbriae, flagella, undesired secretion systems, transposases, effectors, phage elements, or their regulatory elements, such as flhC or OmpA. In some embodiments, an ADAS provided by the invention lacks one or more of an RNAse, a protease, or a combination thereof, and, in particular embodiments, lacks one or more endoribonucleases (such as RNAse A, RNAse h, RNAse III, RNAse L, RNAse PhyM) or exoribonucleases (such as RNAse R, RNAse PH, RNAse D); or serine, cysteine, threonine, aspartic, glutamic and metallo-proteases; or a combination of any of the foregoing.

### F. ADAS Comprising a Targeting Moiety

In another embodiment, the T1P-ADAS comprises a targeting moiety. In some embodiments, the targeting moiety is a nanobody, a carbohydrate binding protein, or a tumor-targeting peptide. In some embodiments, the targeting moiety is an endogenous surface ligand of the parent cell (e.g., a surface ligand that is inherited by the ADAS). In other embodiments, the targeting moiety is an exogenous ligand (e.g., an exogenous tissue targeting ligand) that is added to the ADAS using any of the methods for modifying ADAS described herein. The targeting moiety may promote tissue-associated targeting of the ADAS to a tissue type or cell type.

In certain embodiments, the nanobody is nanobody directed to a tumor antigen, such as HER2, PSMA, or VEGF-R. In other embodiments, the carbohydrate binding protein is a lectin, e.g. Mannose Binding Lectin (MBL). In still other embodiments, the tumor-targeting peptide is an RGD motif or CendR peptide.

### G. ADAS Comprising Additional Moieties

T1P-ADAS provided by the invention can include a variety of additional components, including, for example, photovoltaic pumps, retinals and retinal-producing cassettes, metabolic enzymes, targeting agents, cargo, bacterial secretion systems, and transporters, including combinations of the foregoing, including certain particular embodiments described, below. In certain embodiments, the ADAS lack other elements, such as metabolically non-essential genes and/or certain nucleases or proteases.

A T1P-ADAS, in certain embodiments, includes a functional ATP synthase and, in some embodiments, a membrane embedded proton pump. In some embodiments, a highly active ADAS has an ATP synthase concentration of at least: 1 per 10000 nm2, 1 per 5000 nm2, 1 per 3500 nm2, 1 per 1000 nm2. In certain embodiments, the an ADAS provided by the invention comprises an ATP synthase, optionally lacking a regulatory domain, such as lacking an epsilon domain. Deletion can be accomplished by a variety of means. In certain embodiments, the deletion is by inducible deletion of the native epsilon domain. In certain embodiments, deletion can be accomplished by flanking with LoxP sites and inducible Cre expression or CRISPR knockout, or can be inducible (e.g., place on plasmid under a tTa tet transactivator in an ATP synthase knockout strain)

A TIP-ADAS, in some embodiments, can include a photovoltaic proton pump. In certain embodiments, the photovoltaic proton pump is a proteorhodopsin. In more particular embodiments, the proteorhodopsin comprises the amino acid sequence of proteorhodopsin from the uncultured marine bacterial clade SAR86, GenBank Accession: AAS73014.1. In other embodiments, the photovoltaic proton pump is a gloeobacter rhodopsin. In certain embodiments, the photovoltaic proton pump is a bacteriorhodopsin, deltarhodopsin, or halorhodopsin from Halobium salinarum Natronomonas pharaonis, Exiguobacterium sibiricum, Haloterrigena turkmenica, or Haloarcula marismortui.

In some embodiments, a T1P-ADAS provided by the invention further comprises retinal. In certain embodiments, an ADAS provided by the invention further comprises a retinal synthesizing protein (or protein system), or a nucleic acid encoding the same.

In certain embodiments, a T1P-ADAS provided by the invention further comprises one or more glycolysis pathway proteins. In some embodiments, the glycolysis pathway protein is a phosphofructokinase (Pfk-A), e.g., comprising the amino acid sequence of UniProt accession POA796 or a functional fragment thereof. In other embodiments the glycolysis pathway protein is triosephosphate isomerase (tpi), e.g., comprising the amino acid sequence of UniProt accession POA858, or a functional fragment thereof.

### H. Highly Active ADAS

In some embodiments, the invention provides highly active T1P-ADAS. A “highly active” ADAS is an ADAS with high work potential, e.g. an ADAS having the capability to do a significant amount of useful work. Work may be defined as, e.g., metabolic work, including chemical synthesis (e.g., synthesis of proteins, nucleic acids, lipids, carbohydrates, polymers, or small molecules), chemical modification (e.g., modification of proteins, nucleic acids, lipids, carbohydrates, polymers or small molecules), or transport (e.g., import, export, or secretion) under suitable conditions. In some embodiments, highly active ADAS begin with a large pool of energy, e.g., energy in the form of adenosine triphosphate (ATP). In other embodiments, ADAS have the capacity to take up or generate energy (e.g., ATP) from another source.

In some embodiments, a highly active ADAS has an initial ATP concentration of at least 1 nM, 1.1. nM, 1.2 nM, 1.3 nM, 1.4 mM, 1.5 mM, 1.6 mM, 2 mM, 2.5 mM, 3 nM, 3.5 nM, 4 mM, 5 mM, 10 mM, 20 mM, 30 mM, or 50 mM. ATP concentration can be evaluated by a variety of means including, in certain embodiments, a BacTiter-Glo™ assay (Promega) on lysed ADAS.

High activity may be additionally or alternatively assessed as the rate or amount of increase in ATP concentration in an ADAS over time. In some embodiments, the ATP concentration of an ADAS is increased by at least 50%, at least 60%, at least 75%, at least 100%, at least 150%, at least 200%, or more than 200% following incubation under suitable conditions, e.g., incubation at 37° C. for 12 hours. In certain embodiments, a highly active ADAS has a rate of ATP generation greater than about: 0.000001, 0.00001, 0.0001, 0.001, 0.01, 0.05, 0.1, 0.5, 1.0, 2, 3, 5, 10, 15, 20, 30, 40, 50, 75, 100, 200, 300, 500, 1000, 10000 ATP/see/nm2 for at least about: 1 hour, 2 hours, 3 hours, 4 hours, 5 hours, 6 hours, 12 hours, 1 day, 2 days, 4 days, 1 week, or two weeks.

In other aspects, high activity is assessed as a rate of decrease in ATP concentration over time. In some embodiments, ATP concentration may decrease less rapidly in ADAS that are highly active than in ADAS that are not highly active. In some embodiments, the drop in ATP concentration in an ADAS or an ADAS composition at 24 hours after preparation is less than about 50% (e.g., less than about: 45, 40, 35, 30, 25, 20, 15, 10, or 5%) compared to the initial ATP concentration (e.g., ATP per cell volume), e.g., as measured using a BacTiter-Glo™ assay (Promega).

High activity may be additionally or alternatively assessed as lifetime index of an ADAS. The lifetime index is calculated as the ratio of the rate of GFP production at 24 hours vs. 30 minutes. In some embodiments, a highly active ADAS has a lifetime index of greater than about: 0.13, 0.14, 0.15, 0.16, 0.18, 0.2, 0.25, 0.3,0.35, 0.45, 0.5, 0.60, 0.70, 0.80, 0.90, 1.0 or more. In more particular embodiments, lifetime index is measured in an ADAS containing a functional GFP plasmid with a species-appropriate promoter in which GFP concentration is measured relative to number of ADAS, average number of plasmids per ADAS, and solution volume with a plate reader at 30 minutes and 24 hours.

In some aspects, the ADAS produces a protein, e.g., a heterologous protein. In some aspects, high activity is assessed as a rate, amount, or duration of production of a protein or a rate of induction of expression of the protein (e.g., responsiveness of an ADAS to a signal). For example, the ADAS may comprise a plasmid, the plasmid comprising an inducible promoter and a nucleotide sequence encoding the heterologous protein, wherein contacting the ADAS with an inducer of the inducible promoter under appropriate conditions results in production of the heterologous protein. In some aspects, the production of the heterologous protein is increased by at least 1.6-fold in an ADAS, e.g., a highly active ADAS, that has been contacted with the inducer relative to an ADAS that has not been contacted with the inducer. For example, in some embodiments, the production of the heterologous protein is increased by at least 1.5-fold, 1.75-fold, 2-fold, 3-fold, 4-fold, 5-fold, 10-fold, or more than 10-fold in an ADAS, e.g., a highly active ADAS, that has been contacted with the inducer. In some embodiments, the rate of production of the heterologous protein by a highly active ADAS reaches a target level within a particular duration following the contacting of the ADAS with the inducer, e.g., within 5 minutes, 10 minutes, 15 minutes, 30 minutes, 45 minutes, 1 hour, 2 hours, 3 hours, or more than 3 hours. In some embodiments, a protein (e.g., a heterologous protein) is produced at a rate of at least 0.1 femtograms per hour per highly active ADAS, e.g., at least 0.2 0.4, 0.6, 0.8, 1, 2, 4, 6, 8, 10, 25, 50, 100, 250, 500, 1000, 2000, 3000, or 3500 fg/hour per ADAS. In some embodiments, high activity of an ADAS is assessed as a duration for which a protein is produced. A highly active ADAS may produce a protein (e.g. a heterologous protein) for a duration of at least 2 hours, at least 4 hours, at least 8 hours, at least 12 hours, at least 24 hours, at least 48 hours, or longer than 48 hours.

### I. ADAS Compositions and Formulations

The present invention provides compositions or preparations that contain a T1P-ADAS provided by the invention, including, inter alia, ADAS derived from parent cells that constitutively express T1P components and ADAS derived from parent cells comprising heterologous T1P components. In some embodiments, the ADAS preparation is substantially free of viable cells. Collectively, these are referred to as “compositions provided by the invention” or “a composition provided by the invention”, or the like, and may contain any ADAS provided by the invention and any combination of ADAS provided by the invention.

In some embodiments, a composition provided by the invention comprises a plurality of ADAS, wherein about 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% of the ADAS comprise a T1P.

The compositions provided by the invention can be prepared in any suitable formulation. For example, the formulation can be suitable for IP, IV, IM, oral, topical (cream, gel, ointment, transdermal patch), aerosolized, or nebulized administration. In some embodiments, a formulation is a liquid formulation. In other embodiments, the formulation is a lyophilized formulation.

In some embodiments, an ADAS composition described herein comprises less than 100 colony-forming units (CFU/mL) of viable bacterial cells, e.g., less than 50 CFU/mL, less than 20 CFL/mL, less than 10 CFU/mL, less than 1 CFU/mL, or less than 0.1 CFU/mL of viable bacterial cells.

In some embodiments, the invention provides an ADAS composition wherein the ADAS are lyophilized and reconstituted, and wherein the reconstituted ADAS have a T1P level (e.g., an amount of T1P on the surface of the ADAS or a proportion of ADAS comprising the T1P) that is at least 90% of the T1P level of an ADAS that has not been lyophilized, e.g., at least 95%, 98%, or at least equal to the T1P level of an ADAS that has not been lyophilized.

In some embodiments, the invention provides an ADAS composition wherein the ADAS are stored, e.g., stored at 4° C., wherein after storage, the ADAS have an T1P level that is at least 90% of the T1P level of an ADAS that has not been stored, e.g., at least 95%, 98%, or at least equal to the T1P level of an ADAS that has not been stored. In some embodiments, the storage is for at least one day, at least one week, at least two weeks, at least three weeks, at least one month, at least two months, at least six months, or at least a year.

In some embodiments, ADAS may be preserved or otherwise in a “quiescent” state and then rapidly become activated.

In some embodiments, the ADAS composition is formulated for delivery to a mammal, e.g., formulated for intraperitoneal, intravenous, intramuscular, oral, topical, aerosolized, or nebulized administration. In some embodiments, the composition is formulated for oral delivery.

In some embodiments, the composition is formulated as a liquid, a solid, an aerosol, a paste, a gel, or a gas composition.

### III. Methods of Manufacturing ADAS

### A. Making ADAS

In some aspects, the disclosure features a method for producing an ADAS, the method comprising the steps of (a) providing an engineered bacterium that constitutively expresses the components of a T1P; and (b) producing an ADAS from the bacterium.

Parent bacteria include any suitable bacterial species from which an ADAS may be generated (e.g., species that may be modified using methods described herein to produce ADAS). Table 1 provides a non-limiting list of suitable genera from which ADAS can be derived.

In some aspects, the invention features methods for manufacturing any of the ADAS compositions, e.g., T1P-ADAS compositions, described in Section I herein. For example, provided herein are methods for making ADAS derived from parent cells that constitutively express T1P components, methods for making ADAS derived from parent cells comprising heterologous T1P components, and methods for making any of the ADAS mentioned herein, wherein the ADAS comprises a cargo.

In some embodiments, the ADAS (e.g., T1P-ADAS) is made from a parent strain that is a human bacterium, such as a commensal human bacterium (e.g., E. coli, Staphylococcus sp., Bifidobacterium sp., Micrococcus sp., Lactobacillus sp., or Actinomyces sp.) or a pathogenic human bacterium (e.g., Escherichia coli EHEC, Salmonella typhimurium, Shigella flexneri, Yersinia enterolitica, or Helicobacter pylori), or an extremophile.

In some embodiments, the ADAS and/or parent strain is a functionalized derivative of any of the foregoing, for example including a functional cassette, such as a functional cassette that induces the bacterium to do one or more of: secrete antimicrobials, digest plastic, secrete toxins, survive extreme environments, make nanoparticles, integrate within other organisms, respond to the environment, and create reporter signals.

Parent bacteria may include functionalized derivatives of any of the foregoing, for example including a functional cassette, such as a functional cassette that induces the bacterium to do one or more of: secrete antimicrobials, digest plastic, secrete toxins, survives extreme environments, make nanoparticles, integrate within other organisms, respond to the environment, and create reporter signals.

In some embodiments, an ADAS is derived from a parental strain engineered or induced to overexpress ATP synthase. In some more particular embodiments, the ATP synthase is heterologous to the parental strain. In certain particular embodiments, the parental strain is modified to express a functional F₀F₁ ATP synthase.

In certain embodiments, an ADAS provided by the invention is obtained from a parental strain cultured under a condition selected from: applied voltage (e.g., 37 mV), non-atmospheric oxygen concentration (e.g., 1-5% O₂, 5-10% O₂, 10-15% O₂, 25-30% O₂), low pH (about: 4.5, 5.0, 5.5, 6.0, 6.5), or a combination thereof.

Owing to the diversity of bacterium, ADAS can be made with modified membranes, e.g., to improve the biodistribution of the ADAS upon administration to a target cell. In certain embodiments, the membrane is modified to be less immunogenic or immunostimulatory in mammals. For example, in certain embodiments, the ADAS is obtained from a parental strain, wherein the immunostimulatory capabilities of the parental strain are reduced or eliminated through post-production treatment with detergents, enzymes, or functionalized with PEG. In certain embodiments, the ADAS is made from a parental strain and the membrane is modified through knockout of LPS synthesis pathways in the parental strain, e.g., by knocking out msbB. In other particular embodiments, the ADAS is made from a parental strain that produces cell wall-deficient particles through exposure to hyperosmotic conditions.

In some embodiments, the methods include transforming a parental strain with an inducible DNAse system, such as the exol (NCBI GeneID: 946529) & sbcD (NCBI GeneID: 945049) nucleases, or the I-Ceul (e.g., Swissprot: P32761.1) nuclease. In more particular embodiments, the methods include using a single, double, triple, or quadruple auxotrophic strain and having the complementary genes on the plasmid encoding the inducible nucleases.

In some embodiments, the methods of the methods provided by the invention, the parental strain is cultured under a condition selected from: applied voltage (e.g., 37 mV), non-atmospheric oxygen concentration (e.g., 1-5% O₂, 5-10% O₂, 10-15% O₂, 25-30% O₂), low pH (4.5-6.5), or a combination thereof.

In certain embodiments, the methods of the methods provided by the invention, the parental strain lacks flagella and undesired secretion systems, optionally where the flagella and undesired secretion systems are removed using lambda red recombineering.

In some embodiments, the methods of provided by the invention, flagella control components are excised from the parental strain genome via, for example, insertion of a plasmid containing a CRISPR domain that is targeted towards flagella control genes, such as flhD and flhC.

In certain embodiments, the methods provided by the invention are for making a highly active ADAS, where an ADAS comprising a plasmid containing a rhodopsin-encoding gene is cultured in the presence of light. In more particular embodiments, the rhodopsin is proteorhodopsin from SAR86 uncultured bacteria, having the amino acid sequence of GenBank Accession: AAS73014.1, or a functional fragment thereof. In still more particular embodiments, the culture is supplemented with retinal. In other more particular embodiments, the rhodopsin is proteorhodopsin and the plasmid additionally contains genes synthesizing retinal (such a plasmid is the pACYC-RDS plasmid from Kim et al., Microb Cell Fact, 2012).

In certain particular embodiments, the parental strain contains a nucleic acid sequence encoding a nanobody that is then expressed on the membrane of the ADAS.

In some embodiments of the methods provided by the invention, the parental strain contains a nucleic acid sequence encoding one or more bacterial secretion system operons. Exemplary plasmids include the Salmonella SPI-1 T3SS, the Shigella flexneri T3SS, the Agrobacterium Ti plasmid, and the Pseudomonas putida K1-T6SS system.

In certain embodiments, the parental strain comprises a cargo. In some embodiments, the parent strain contains a nucleic acid sequence encoding a set of genes that synthesize a small molecule cargo.

### IV. Purification of Adas and Adas Compositions

In some embodiments of the methods and compositions provided herein, ADAS are purified from compositions (e.g., cultures) comprising viable bacteria, e.g., parental bacteria. An exemplary method for manufacturing a composition comprising a plurality of ADAS, the composition being substantially free of viable bacterial cells, comprises (a) making, providing, or obtaining a plurality of parent bacteria having a reduction in the level or activity of a cell division topological specificity factor; (b) exposing the parent bacteria to conditions allowing the formation of a minicell, thereby producing the ADAS; and (c) separating the ADAS from the parent bacteria, thereby producing a composition that is substantially free of viable bacterial cells.

Purification separates ADAS from viable parent bacterial cells, which contain a genome and may be larger. Separating the highly active ADAS from the parent bacteria can be performed using a number of methods, as described herein. Exemplary methods for purification described herein include centrifugation, selective outgrowth, and buffer exchange/concentration processes.

In some aspects, provide herein are ADAS compositions, and methods of comparing such compositions, wherein the compositions are substantially free of parent bacterial cells and/or viable bacterial cells, e.g., have no more than 500, e.g., 400, 300, 200, 150, or 100 or fewer than 50, fewer than 25, fewer than 10, fewer than 5, fewer than 1, fewer than 0.1 colony-forming units (CFU) per mL. In some embodiments, an ADAS composition that is substantially free of parent bacterial cells may include no bacterial cells.

Auxotrophic parental strains can be used to make ADAS provided by the invention. As described in more detail below, such manufacturing methods are useful for purification of the ADAS. For example, following ADAS generation, parent bacterial cells may be removed by growth in media lacking the nutrient (for example, amino acid) necessary for viability of the parent bacterium. In some embodiments, an ADAS provided by the invention is derived from a parental strain auxotrophic for at least 1, 2, 3, 4, or more of: arginine (e.g., knockout in argA, such as strains JW2786-1 and NK5992), cysteine knockout in cysE (such as strains JW3582-2 and JM15), glutamine e.g., knockout in glnA (such as strains JW3841-1 and M5004), glycine e.g., knockout in glyA (such as strains JW2535-1 and AT2457), Histidine e.g., knockout in hisB (such as strains JW2004-1 and SB3930), isoleucine e.g., knockout in ilvA (such as strains JW3745-2 and AB1255), leucine e.g., knockout in leuB (such as strains JW5807-2 and CV514), lysine e.g., knockout in lysA (such as strains JW2806-1 and KL334), methionine e.g., knockout in metA (such as strains JW3973-1 and DL41), phenylalanine e.g., knockout in pheA (such as strains JW2580-1 and KA197), proline e.g., knockout in proA (such as strains JW0233-2 and NK5525), Serine e.g., knockout in serA (such as strains JW2880-1 and JC158), threonine e.g., knockout in thrC (such as strains JW0003-2 and Gif 41), tryptophan e.g., knockout in trpC (such as strains JW1254-2 and CAG18455), Tyrosine e.g., knockout in tyrA (such as strains JW2581-1 and N3087), Valine/Isoleucine/Leucine e.g., knockout in ilvd (such as strains JW5605-1 and CAG18431).

In certain embodiments, the methods include using a single, double, triple, or quadruple auxotrophic parental strain, optionally wherein said parental strain further includes a plasmid expressing a ftsZ.

### V. Methods of Using ADAS

A. Methods of delivering an ADAS

In some aspects, the disclosure features a method for delivering an ADAS to a cell, the method comprising (a) providing a composition comprising a plurality of the ADAS of the invention (e.g., T1P-ADAS); and (b) contacting the cell with the composition of step (a). In some embodiments, delivery of the ADAS to the cell is increased by at least 1% relative to an ADAS derived from a control (e.g., unmodified) parent cell (e.g., increased by at least 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., increased by 1%-5%, 5%-10%, 10%-20%, 20%-30%, 30%-40%, 40%-50%, 50%-60%, 60%-70%, 70%-80%, 80%-90%, 90%-100%, or more than 100% relative to an ADAS derived from a control parent cell). In some aspects, delivery of the ADAS to the cell is increased by at least 10% relative to an ADAS derived from a control parent cell.

In some embodiments, an effective amount of the ADAS is delivered to the cell at a dose that is at least 1% lower than the dose required for an ADAS derived from a control parent cell (e.g., at least 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., increased by 1%-5%, 5%-10%, 10%-20%, 20%-30%, 30%-40%, 40%-50%, 50%-60%, 60%-70%, 70%-80%, 80%-90%, or 90%-99% lower than the dose required for an ADAS derived from a control parent cell). In some embodiments, an effective amount of the ADAS is delivered to the cell at a dose that is at least 10% lower than the dose required for an ADAS derived from a control parent cell.

In some embodiments, the cell is a mammalian cell (e.g., a human cell or a non-human mammal cell, including a cell of a farm animal, a domestic animal, or a pest animal). In some embodiments, the mammalian cell is a gut cell, e.g., a gut-associated lymphoid tissue (GALT) cell, a Peyer's patch cell, an M cell, a lamina propria cell, a small intestine cell, or a large intestine cell. In other embodiments, the mammalian cell is a bladder cell, an immune cell, or a blood-brain barrier cell. In some embodiments, the mammalian cell is a mannosylated cell.

### B. Methods of Delivering a Cargo

In some aspects, the disclosure features a method for delivering a cargo to a cell, the method comprising (a) providing a composition comprising a plurality of the ADAS of the invention (e.g., T1P-ADAS), wherein the ADAS further comprise a cargo; and (b) contacting the cell with the composition of step (a).

In some embodiments, the ADAS further comprise a heterologous bacterial secretion system. In some embodiments, the heterologous bacterial secretion system is a T3SS. In some embodiments, the delivery is to the cytoplasm of the cell.

In some embodiments, delivery of the cargo to the cell is increased by at least 1% relative to an ADAS derived from a control (e.g., unmodified) parent cell (e.g., increased by at least 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., increased by 1%-5%, 5%-10%, 10%-20%, 20%-30%, 30%-40%, 40%-50%, 50%-60%, 60%-70%, 70%-80%, 80%-90%, 90%-100%, or more than 100% relative to an ADAS derived from a control parent cell). In some embodiments, delivery of the cargo to the cell is increased by at least 10% relative to an ADAS derived from a control parent cell.

In some embodiments, an effective amount of the cargo is delivered to the cell at a dose that is at least 1% lower than the dose required for an ADAS derived from a control (e.g., unmodified) parent cell (e.g., at least 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., increased by 1%-5%, 5%-10%, 10%-20%, 20%-30%, 30%-40%, 40%-50%, 50%-60%, 60%-70%, 70%-80%, 80%-90%, or 90%-99% lower than the dose required for an ADAS derived from a control parent cell). In some embodiments, an effective amount of the cargo is delivered to the cell at a dose that is at least 10% lower than the dose required for an ADAS derived from a control parent cell.

In some embodiments, the cell is a mammalian cell (e.g., a human cell or a non-human mammal cell, including a cell of a farm animal, a domestic animal, or a pest animal). In some embodiments, the mammalian cell is a gut cell, e.g., a gut-associated lymphoid tissue (GALT) cell, a Peyer's patch cell, an M cell, a lamina propria cell, a small intestine cell, or a large intestine cell. In other embodiments, the mammalian cell is a bladder cell, an immune cell, or a blood-brain barrier cell. In some embodiments, the mammalian cell is a mannosylated cell.

### C. Methods of Modulating a Cell

In some aspects, the disclosure features a method of modulating a cell, the method comprising (a) providing a composition comprising a plurality of the ADAS of the invention (e.g., T1P-ADAS); and (b) contacting the cell with the composition of step (a), whereby the cell is modulated.

In some embodiments, the cell is a mammalian cell (e.g., a human cell or a non-humanmammal cell, including a cell of a farm animal, a domestic animal, or a pest animal). In some embodiments, the mammalian cell is a gut cell, e.g., a gut-associated lymphoid tissue (GALT) cell, a Peyer's patch cell, an M cell, a lamina propria cell, a small intestine cell, or a large intestine cell. In other embodiments, the mammalian cell is a bladder cell, an immune cell, or a blood-brain barrier cell. In some embodiments, the mammalian cell is a mannosylated cell.

The modulating may be any observable change in the state (e.g., the transcriptome, proteome, epigenome, biological effect, or health or disease state) of the cell (e.g. mammalian cell) as measured using techniques and methods known in the art for such a measurement, e.g., methods to measure the level or expression of a protein, a transcript, an epigenetic mark, or to measure the increase or reduction of activity of a biological pathway. In some embodiments, modulating the state of the cell involves increasing a parameter (e.g., the level or expression of a protein, a transcript, or activity of a biological pathway) of the cell. In other embodiments, modulating the state of involves decreasing a parameter (e.g., the level or expression of a protein, a transcript, or activity of a biological pathway) of the cell.

The cell may be isolated (e.g. in vitro) or within an organism (e.g., in vivo). The methods provided herein entail providing an ADAS provided by the invention or a composition provided by the invention with access to the target cell, in an effective amount. The access to the target cell may either be direct (e.g., the target cell is modulated directly by the ADAS, such as by proximate secretion of an agent (e.g., a cargo of the ADAS) proximate to the target cell or injection of the agent (e.g., cargo) into the target cell) or indirect. Indirect modulation of the target cell may be by targeting a different cell, for example, by modulating a cell adjacent to the target cell, which adjacent cell may be commensal or pathogenic to the target cell. The adjacent cell, like the target cell, may be either in vitro or in vivo (e.g., in an organism, which may be commensal or pathogenic). These methods are collectively “methods of use provided by the invention” and the like. In a related aspect, the invention provides target uses of the ADAS and compositions provided by the invention, consonant with the methods of use provided by the invention.

For example, in some embodiments, the invention provides method of modulating a state of a mammalian cell, by providing an effective amount of an ADAS provided by the invention or composition provided by the invention access to the mammalian cell. In certain embodiments, the ADAS or composition is provided access to the mammalian cell in vivo, in a mammal (e.g., a human). In some embodiments, the mammalian cell is exposed to bacteria in a healthy mammal. In more particular embodiments, the mammalian cell is lung epithelium, an immune cell, skin cell, oral epithelial cell, gut epithelial cell, reproductive tract epithelial cell, or urinary tract cell. In still more particular embodiments, the mammalian cell is a gut epithelial cell, such as a gut epithelial cell from a human subject with an inflammatory bowel disease, such as Crohn's disease or colitis. In yet more particular embodiments, the mammalian cell is a gut epithelial cell from a subject with an inflammatory bowel disease, and the ADAS comprises a bacterial secretion system and a cargo comprising an anti-inflammatory agent.

In other embodiments the mammalian cell is exposed to bacteria in a diseased state. In certain embodiments, the mammalian cell is pathogenic, such as a tumor. In other embodiments, the mammalian cell is exposed to bacteria in a diseased state such as a wound, an ulcer, a tumor, or an inflammatory disorder

In certain embodiments, the ADAS is derived from an mammal commensal parental strain. In other embodiments, the ADAS is derived from a mammal pathogenic parental strain.

In some embodiments, the state of the mammalian cell is modulated by providing an effective amount of an ADAS provided by the invention or a composition provided by the invention with access to a bacterial or fungal cell in the vicinity of the mammalian cell. That is, these methods entail indirectly modulating the state of the mammalian cell. In certain embodiments, the bacterial or fungal cell is pathogenic. In more particular embodiments, the fitness of the pathogenic bacterial or fungal cell is reduced. In other certain embodiments, the bacterial or fungal cell is commensal. In more particular embodiments, the fitness of the commensal bacterial or fungal cell is increased. In still more particular embodiments, the fitness of the commensal bacterial or fungal strain is increased via reduction in fitness of number of a competing bacteria or fungi, which may be neutral, commensal, or pathogenic.

### D. Methods of Treating a Mammal

In some aspects, the disclosure features a method of treating a mammal (e.g., a human or a non-human mammal, e.g., a farm animal, a domestic animal, or a pest animal) in need thereof, the method comprising (a) providing a composition comprising a plurality of the ADAS of the invention (e.g., T1P-ADAS); and (b) contacting the mammal with an effective amount of the composition of step (a), thereby treating the mammal.

In some embodiments, a therapeutic effect is achieved at a dose that is at least 1% lower than the dose required for an ADAS derived from a control parent cell (e.g., unmodified) (e.g., at least 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., increased by 1%-5%, 5%-10%, 10%-20%, 20%-30%, 30%-40%, 40%-50%, 50%-60%, 60%-70%, 70%-80%, 80%-90%, or 90%-99% lower than the dose required for an ADAS derived from a control parent cell). In some embodiments, a therapeutic effect is achieved at a dose that is at least 10% lower than the dose required for an ADAS derived from a control parent cell.

The mammal in need of treatment may have a disease, e.g., a cancer. In some embodiments, the ADAS carries a chemotherapy cargo or an immunotherapy cargo.

### OTHER EMBODIMENTS

The invention is further described in the following numbered paragraphs:

1. A plurality of achromosomal dynamic active systems (ADAS) comprising a type 1 pilus (T1P), wherein the ADAS are derived from parent bacterial cells that constitutively express the components of the T1P.

2. The plurality of ADAS of paragraph 1, wherein the parent cells comprise a modified fimS promoter that is operably linked to, and directs constitutive expression of, the components of the T1P.

3. The plurality of ADAS of paragraph 1 or 2, wherein the components of the T1P are encoded by a fim operon.

4. The plurality of ADAS of paragraph 3, wherein the parent cells comprise a modified fimS promoter operably linked to the fim operon, wherein the modified fimS promoter comprises a mutation at a recombinase cleavage site that prevents recombination of the fimS promoter into an ‘OFF’ orientation.

5. The plurality of ADAS of any one of paragraphs 1-4, wherein the parent cells express the components of the T1P at a level that is at least 1.5-fold greater than a level observed in an unmodified parent cell.

6. The plurality of ADAS of any one of paragraphs 1-5, wherein the ADAS comprise the T1P at a level that is at least 1.5-fold greater than a level observed in a plurality of ADAS produced from unmodified parent cells.

7. The plurality of ADAS of any one of paragraphs 1-6, wherein the proportion of the plurality of ADAS comprising a T1P is increased relative to a plurality of ADAS produced by parent cells that do not constitutively express the components of a T1P.

8. The plurality of ADAS of any one of paragraphs 1-7, wherein the parent cells comprise an endogenous fim operon.

9. The plurality of ADAS of any one of paragraphs 1-8, wherein the parent cells are E. coli bacteria.

10. The plurality of ADAS of paragraph 9, wherein the E. coli bacteria are E. coli CFT073.

11. The plurality of ADAS of any one of paragraphs 1-10, wherein the parent cells comprise one or more heterologous nucleotide sequences encoding the components of the T1P.

12. The plurality of ADAS of paragraph 11, wherein the one or more heterologous nucleotide sequences comprise a fim operon.

13. The plurality of ADAS of paragraph 12, wherein the fim operon is the fim operon of E. coli CFT073.

14. The plurality of ADAS of paragraph 13, wherein the one or more heterologous nucleotide sequences comprise a sequence having at least 90% identity to the nucleotide sequence of SEQ ID NO: 1.

15. The plurality of ADAS of paragraph 14, wherein the one or more heterologous nucleotide sequences comprise a sequence having at least 95% identity to the nucleotide sequence of SEQ ID NO: 1.

16. The plurality of ADAS of paragraph 15, wherein the one or more heterologous nucleotide sequences comprise the nucleotide sequence of SEQ ID NO: 1.

17. The ADAS of any one of paragraphs 12-16, wherein the one or more heterologous nucleotide sequences further comprise a constitutive promoter operably linked to the fim operon.

18. The plurality of ADAS of paragraph 17, wherein the constitutive promoter is a modified fimS promoter comprising a mutation at a recombinase cleavage site that prevents recombination of the fimS promoter into an ‘OFF’ orientation.

19. The plurality of ADAS of any one of paragraphs 11-18, wherein the one or more nucleotide sequences encoding the components of the T1P are carried on a vector.

20. The plurality of ADAS of paragraph 19, wherein the parent bacterial cells have been transiently transformed with the vector.

21. The plurality of ADAS of paragraph 19, wherein the parent bacterial cells have been stably transformed with the vector.

22. The plurality of ADAS of any one of paragraphs 10-21, wherein the parent cells are Gram-negative bacterial cells.

23. The plurality of ADAS of paragraph 22, wherein the Gram-negative bacterial cells are E. coli, Salmonella, Yersinia, Vibrio, Pseudomonas, Shigella, or Legionella bacterial cells.

24. The plurality of ADAS of any one of paragraphs 11-23, wherein the parent cells do not comprise a complete endogenous fim operon.

25. The plurality of ADAS of any one of paragraphs 3, 4, and 12-24, wherein the parent cells have not been exposed to a culture condition that promotes the expression of the fim operon.

26. The plurality of ADAS of paragraph 25, wherein the culture condition is temperature, pH, osmolality, shaking, or activation of the stress or stringent response.

27. The plurality of ADAS of any one of paragraphs 1-26, wherein the ADAS comprise a cargo.

28. The plurality of ADAS of paragraph 27, wherein the cargo is a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP).

29. The plurality of ADAS of paragraph 27 or 28, wherein the cargo is encapsulated by the ADAS.

30. The plurality of ADAS of paragraph 27 or 28, wherein the cargo is attached to the surface of the ADAS.

31. The plurality of ADAS of any one of paragraphs 1-30, wherein the ADAS comprise a heterologous bacterial secretion system.

32. The plurality of ADAS of paragraph 31, wherein the heterologous bacterial secretion system is a type 3 secretion system (T3SS).

33. The plurality of ADAS of paragraph 31 or 32, wherein the cargo comprises a moiety that directs export by the bacterial secretion system.

34. A composition comprising the plurality of ADAS of any one of paragraphs 1-33.

35. The composition of paragraph 34, wherein the composition is formulated for delivery to a mammal.

36. The composition of paragraph 35, wherein the composition is formulated for oral delivery.

37. A method for delivering an ADAS to a cell, the method comprising:
(a) providing a composition comprising the plurality of ADAS of any one of paragraphs 1-36; and
(b) contacting the cell with the composition of step (a).

38. The method of paragraph 37, wherein delivery of the ADAS to the cell is increased by at least 10% relative to an ADAS derived from an unmodified parent cell.

39. The method of paragraph 37 or 38, wherein an effective amount of the ADAS is delivered to the cell at a dose that is at least 10% lower than the dose required for an ADAS derived from an unmodified parent cell.

40. A method for delivering a cargo to a cell, the method comprising:
(a) providing a composition comprising a plurality of the ADAS of any one of paragraphs 1-36, wherein the ADAS further comprise a cargo; and
(b) contacting the cell with the composition of step (a).

41. The method of any one of paragraphs 37-40, wherein the ADAS further comprise a heterologous bacterial secretion system.

42. The method of paragraph 41, wherein the heterologous bacterial secretion system is a T3SS.

43. The method of any one of paragraphs 37-42, wherein the delivery is to the cytoplasm of the cell.

44. The method of any one of paragraphs 40-43, wherein delivery of the cargo to the cell is increased by at least 10% relative to an ADAS derived from an unmodified parent cell.

45. The method of any one of paragraphs 40-43, wherein an effective amount of the cargo is delivered to the cell at a dose that is at least 10% lower than the dose required for an ADAS derived from an unmodified parent cell.

46. A method of modulating a cell, the method comprising:
(a) providing a composition comprising the plurality of ADAS of any one of paragraphs 1-36; and
(b) contacting the cell with the composition of step (a), whereby the cell is modulated. 47. The method of paragraph any one of paragraphs 37-46, wherein the cell is a mammalian cell.

48. The method of paragraph 47, wherein the mammalian cell is a gut cell.

49. The method of paragraph 48, wherein the gut cell is a gut-associated lymphoid tissue (GALT) cell, a Peyer's patch cell, an M cell, a lamina propria cell, a small intestine cell, or a large intestine cell.

50. The method of paragraph 47, wherein the mammalian cell is a bladder cell.

51. The method of paragraph 47, wherein the mammalian cell is an immune cell.

52. The method of paragraph 47, wherein the mammalian cell is a blood-brain barrier cell.

53. The method of any one of paragraphs 47-52, wherein the mammalian cell is a mannosylated cell.

54. A method of treating a mammal in need thereof, the method comprising:
(a) providing a composition comprising the plurality of ADAS of any one of paragraphs 1-36; and
(b) contacting the mammal with an effective amount of the composition of step (a), thereby treating the mammal.

55. The method of paragraph 54, wherein a therapeutic effect is achieved at a dose that is at least 10% lower than the dose required for an ADAS derived from an unmodified parent cell.

56. An ADAS comprising a T1P derived from a parent bacterial cell, wherein the ADAS is produced by a process comprising the steps of:
(a) providing a parent cell that has been modified to constitutively express the components of a T1P; and
(b) producing an ADAS from the parent bacterial cell, wherein the ADAS comprises the T1P.

57. An ADAS comprising a T1P derived from a parent bacterial cell, wherein the ADAS is produced by a process comprising the steps of:
(a) providing a parent cell that has been modified to express the components of a native T1P at a level that is at least 1.5-fold greater than a level observed in an unmodified parent cell; and
(b) producing an ADAS from the parent bacterial cell, wherein the ADAS comprises the T1P that is native to the parent cell.

58. An ADAS comprising a T1P derived from a parent bacterial cell, wherein the ADAS is produced by a process comprising the steps of:
(a) providing a parent cell that has been modified to express the components of a heterologous T1P; and
(b) producing an ADAS from the parent bacterial cell, wherein the ADAS comprises the T1P that is heterologous to the parent cell.

59. An engineered bacterium constitutively expressing the components of a T1P, wherein the engineered bacterium comprises the T1P at a level which is at least 1.5-fold greater compared to the level of T1P comprised by a non-engineered bacterium.

60. The bacterium of paragraph 59, wherein the T1P is a native T1P.

61. The bacterium of paragraph 59, wherein the T1P is a heterologous T1P.

62. An engineered bacterium that constitutively expresses the components of a T1P, wherein the bacterium has been modified to produce ADAS.

63. A method for producing an ADAS, the method comprising the steps of:
(a) providing an engineered bacterium that constitutively expresses the components of a T1P; and
(b) producing an ADAS from the bacterium.

64. An ADAS produced according to the method of paragraph 63.

The invention is further described in the following numbered paragraphs:

1. A preparation comprising a plurality of achromosomal dynamic active systems (ADAS) derived from parent bacterial cells genetically engineered to constitutively express a type 1 pilus (T1P), wherein the plurality of ADAS binds a target cell via the T1P.

2. The preparation of paragraph 1, wherein the parent bacterial cells comprise a modified fimS promoter that is operably linked to, and directs constitutive expression of, the components of the T1P.

3. The preparation of paragraph 1 or 2, wherein the components of the T1P are encoded by a fim operon.

4. The preparation of paragraph 3, wherein the parent bacterial cells comprise a modified fimS promoter operably linked to the fim operon, wherein the modified fimS promoter comprises a mutation at a recombinase cleavage site that prevents recombination of the fimS promoter into an ‘OFF’ orientation.

5. The preparation of any one of paragraphs 1-4, wherein the parent bacterial cells express the components of the T1P at a level that is at least 1.5-fold greater than a level observed in an unmodified parent bacterial cell.

6. The preparation of any one of paragraphs 1-5, wherein the plurality of ADAS comprise the T1P at a level that is at least 1.5-fold greater than a level observed in a plurality of ADAS produced from unmodified parent bacterial cells.

7. The preparation of any one of paragraphs 1-6, wherein the proportion of the plurality of ADAS comprising a T1P is increased relative to a plurality of ADAS produced by parent bacterial cells that do not constitutively express the components of a T1P.

8. The preparation of any one of paragraphs 1-7, wherein the parent bacterial cells comprise an endogenous fim operon.

9. The preparation of any one of paragraphs 1-8, wherein the parent bacterial cells are E. coli bacteria.

10. The preparation of paragraph 9, wherein the E. coli bacteria are E. coli CFT073.

11. The preparation of any one of paragraphs 1-10, wherein the parent bacterial cells comprise one or more heterologous nucleotide sequences encoding the components of the T1P.

12. The preparation of paragraph 11, wherein the one or more heterologous nucleotide sequences comprise a fim operon.

13. The preparation of paragraph 12, wherein the fim operon is the fim operon of E. coli CFT073.

14. The preparation of paragraph 13, wherein the one or more heterologous nucleotide sequences comprise a sequence having at least 90% identity to the nucleotide sequence of SEQ ID NO: 1.

15. The preparation of paragraph 14, wherein the one or more heterologous nucleotide sequences comprise a sequence having at least 95% identity to the nucleotide sequence of SEQ ID NO: 1.

16. The preparation of paragraph 15, wherein the one or more heterologous nucleotide sequences comprise the nucleotide sequence of SEQ ID NO: 1.

17. The preparation of any one of paragraphs 12-16, wherein the one or more heterologous nucleotide sequences further comprise a constitutive promoter operably linked to the fim operon.

18. The preparation of paragraph 17, wherein the constitutive promoter is a modified fimS promoter comprising a mutation at a recombinase cleavage site that prevents recombination of the fimS promoter into an ‘OFF’ orientation.

19. The preparation of any one of paragraphs 11-18, wherein the one or more nucleotide sequences encoding the components of the T1P are carried on a vector.

20. The preparation of paragraph 19, wherein the parent bacterial cells have been transiently transformed with the vector.

21. The preparation of paragraph 19, wherein the parent bacterial cells have been stably transformed with the vector.

22. The preparation of any one of paragraphs 10-21, wherein the parent bacterial cells are Gram-negative bacterial cells.

23. The preparation of paragraph 22, wherein the Gram-negative bacterial cells are E. coli, Salmonella, Yersinia, Vibrio, Pseudomonas, Shigella, or Legionella bacterial cells.

24. The preparation of any one of paragraphs 11-23, wherein the parent bacterial cells do not comprise a complete endogenous fim operon.

25. The preparation of any one of paragraphs 3, 4, and 12-24, wherein the parent bacterial cells have not been exposed to a culture condition that promotes the expression of the fim operon.

26. The preparation of paragraph 25, wherein the culture condition is temperature, pH, osmolality, shaking, or activation of the stress or stringent response.

27. The preparation of any one of paragraphs 1-26, wherein the ADAS comprise a cargo.

28. The preparation of paragraph 27, wherein the cargo is a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP).

29. The preparation of paragraph 27 or 28, wherein the cargo is encapsulated by the ADAS.

30. The preparation of paragraph 27 or 28, wherein the cargo is attached to the surface of the ADAS.

31. The preparation of any one of paragraphs 1-30, wherein the ADAS comprise a heterologous bacterial secretion system.

32. The preparation of paragraph 31, wherein the heterologous bacterial secretion system is a type 3 secretion system (T3SS) or a type 6 secretion system (T6SS).

33. The preparation of paragraph 31 or 32, wherein the cargo comprises a moiety that directs export by the bacterial secretion system.

34. A composition comprising the preparation of a plurality of ADAS of any one of paragraphs 1-33.

35. The composition of paragraph 34, wherein the composition is formulated for delivery to a mammal.

36. The composition of paragraph 35, wherein the composition is formulated for oral delivery.

37. A method for delivering an ADAS to a cell, the method comprising contacting a cell with a composition comprising the preparation of a plurality of ADAS of any of paragraphs 1-36.

38. The method of paragraph 37, wherein delivery of the ADAS to the cell is increased by at least 10% relative to an ADAS derived from an unmodified parent bacterial cell.

39. The method of paragraph 37 or 38, wherein an effective amount of the ADAS is delivered to the cell at a dose that is at least 10% lower than the dose required for an ADAS derived from an unmodified parent bacterial cell.

40. A method for delivering a cargo to a cell, the method comprising contacting a cell with a composition comprising the preparation of a plurality of the ADAS of any one of paragraphs 1-36, wherein the ADAS further comprise a cargo.

41. The method of any one of paragraphs 37-40, wherein the ADAS further comprise a heterologous bacterial secretion system.

42. The method of paragraph 41, wherein the heterologous bacterial secretion system is a T3SS or T6SS.

43. The method of any one of paragraphs 37-42, wherein the delivery is to the cytoplasm of the cell.

44. The method of any one of paragraphs 40-43, wherein delivery of the cargo to the cell is increased by at least 10% relative to an ADAS derived from an unmodified parent bacterial cell.

45. The method of any one of paragraphs 40-43, wherein an effective amount of the cargo is delivered to the cell at a dose that is at least 10% lower than the dose required for an ADAS derived from an unmodified parent bacterial cell.

46. A method of modulating a cell, the method comprising contacting a cell with a composition comprising the preparation of a plurality of ADAS of any one of paragraphs 1-36, whereby the cell is modulated.

47. The method of paragraph any one of paragraphs 37-46, wherein the cell is a mammalian cell.

48. The method of paragraph 47, wherein the mammalian cell is a gut cell.

49. The method of paragraph 48, wherein the gut cell is a gut-associated lymphoid tissue (GALT) cell, a Peyer's patch cell, an M cell, a lamina propria cell, a small intestine cell, or a large intestine cell.

50. The method of paragraph 47, wherein the mammalian cell is a bladder cell.

51. The method of paragraph 47, wherein the mammalian cell is an immune cell.

52. The method of paragraph 47, wherein the mammalian cell is a blood-brain barrier cell.

53. The method of any one of paragraphs 47-52, wherein the mammalian cell is a mannosylated cell.

54. An ADAS derived from a parent bacterial cell genetically engineered to constitutively express a T1P, wherein the ADAS is produced by a process comprising the steps of:
(a) providing a parent bacterial cell that has been modified to constitutively express the components of a T1P; and
(b) producing an ADAS from the parent bacterial cell, wherein the ADAS comprises the T1P.

55. An ADAS derived from a parent bacterial cell genetically engineered to constitutively express a T1P, wherein the ADAS is produced by a process comprising the steps of:
(a) providing a parent bacterial cell that has been modified to express the components of a native T1P at a level that is at least 1.5-fold greater than a level observed in an unmodified parent bacterial cell; and
(b) producing an ADAS from the parent bacterial cell, wherein the ADAS comprises the T1P that is native to the parent bacterial cell.

56. An ADAS derived from a parent bacterial cell genetically engineered to constitutively express a T1P, wherein the ADAS is produced by a process comprising the steps of:
(a) providing a parent bacterial cell that has been modified to express the components of a heterologous T1P; and
(b) producing an ADAS from the parent bacterial cell, wherein the ADAS comprises the T1P that is heterologous to the parent bacterial cell.

57. A genetically engineered bacterium that constitutively expresses the components of a T1P, wherein the engineered bacterium comprises the T1P at a level which is at least 1.5-fold greater compared to the level of T1P comprised by a non-engineered bacterium.

58. The bacterium of paragraph 57, wherein the T1P is a native T1P.

59. The bacterium of paragraph 57, wherein the T1P is a heterologous T1P.

60. A genetically engineered bacterium that constitutively expresses the components of a T1P, wherein the bacterium has been modified to produce ADAS.

61. A method for producing an ADAS, the method comprising producing an ADAS from a bacterium genetically engineered to constitutively expresses the components of a T1P.

62. An ADAS produced according to the method of paragraph 61.

### EXAMPLES

### Example 1: Methods for Manufacturing ADAS Expressing Endogenous or Heterologous Type 1 Pili (T1P-ADAS)

### A. ADAS Expressing Endogenous T1P

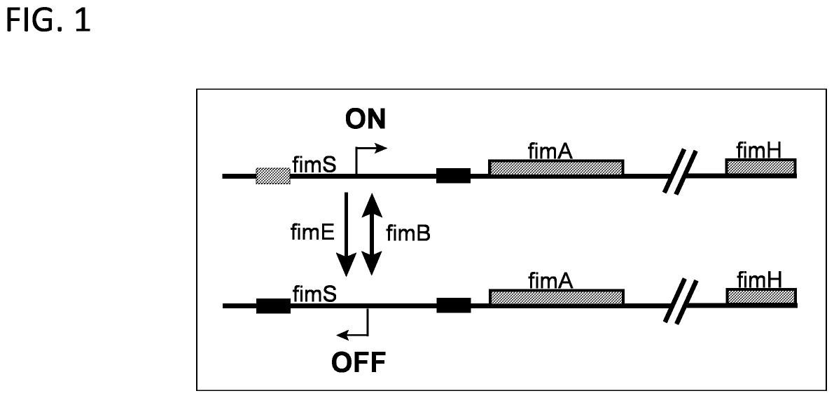

The native promoter for the type 1 pilus (T1P), fimS, is phase variable. Phase variable promoters are pieces of DNA that can be inverted after cleavage by site-specific recombinases. The recombinases fimB and fimE bind and cut at inverted repeat sites (IR sites; shown as black boxes in FIG. 1) flanking fimS and can flip the promoter into an ‘ON’ orientation (promoter faces the operon; fim operon is expressed) or an ‘OFF’ orientation (promoter faces away from the operon, no expression of fim operon) (FIG. 1). The fimS expression state is heritable, but also reversible, and inversion is influenced by numerous signals, including temperature, pH, osmolality, and the stress and stringent responses. In the lab, growing E. coli in LB broth at 37° C., shaking (the standard growth condition for E. coli) promotes maintenance of the fimS switch in the ‘OFF’ orientation. Switching growth to 37° C., static (rather than shaking) promotes maintenance of the fimS switch in the ‘ON’ orientation.

The recombinase cleavage site upstream of the promoter (green box in FIG. 1) was mutated using Lambda-RED recombineering, locking it into the ON orientation (“locked-on” (“LON”) operon), thus generating bacteria that constitutively express the fim operon (i.e., constitutively express T1P). This was done in two E. coli strains: a lab-adapted strain (BW25113) and a pathogenic strain (CFT073). The bacteria were further engineered to produce ADAS by deleting the minCDE locus using Lambda-RED recombineering, as described in WO 2020/123569.

The wild-type sequences (5′->3′) of the fimS sites in the E. coli strains CFT073 and MG1655 with the fimS promoter in the ‘ON’ orientation are shown in SEQ ID NO: 4 and SEQ ID NO: 6, respectively. The left inverted repeat site is underlined and italicized. SEQ ID NO: 5 and SEQ ID NO: 7, shown below, show mutations made to the left inverted repeat site. Mutations are indicated by capital letters. While very similar, the fimS operons from CFT073 and MG1655 have some variation in the promoter sequence. The inverted repeat site sequences and the mutations made to lock the promoter ‘on’ are identical.

CFT073 fimS natural ‘on’ orientation
(SEQ ID NO: 4)
ttggggccattttgactcatagaggaaagcatcgcgcacaaactttttcagtttatttgttggcttaatgttctatagttatctttatttg
cagttttttatattgcatgaggtggtttttggagagaagaatgaggaagttgcgtcgagctacagaaacgttagctttacatatagcggagg
tgatgtgaaattaatttacaagagaaataatttacatatcaaacagttagatgctttttgtcgttttttaatatttttatgcttgagaaaaa
atacgtaacttatttatgatatagacagtttggccccaa
CFT073 fimS ‘Locked on’ sequence. Mutation from natural sequence in bolded, in caps.
(SEQ ID NO: 5)
ttAgATcTCttttgactcatagaggaaagcatcgcgcacaaactttttcagtttatttgttggcttaatgttctatagttatctttatttg
cagttttttatattgcatgaggtggtttttggagagaagaatgaggaagttgcgtcgagctacagaaacgttagctttacatatagcggagg
tgatgtgaaattaatttacaagagaaataatttacatatcaaacagttagatgctttttgtcgttttttaatatttttatgcttgagaaaa
aatacgtaacttatttatgatatagacagtttggccccaa
MG1655 fimS natural ‘on’ orientation
(SEQ ID NO: 6)
ttggggccattttgactcatagaggaaagcatcgcggacaaactttttcagtttatttgttggcttaatattctattgttatctttattta
tagatgtttatattgcatgaggtggtttttggagagaagaatgaggaagatgcgtcgagccacagaaacgttagctttacatatagcggag
gtgatgtgaaattaatttacaatagaaataatttacatatcaaacagttagatgctttttgtcgttttttaatatttttatgcttgagaaa
aaatacgtaacttatttatgatatggacagtttggccccaa
MG1655 fimS ‘Locked on’ orientation
(SEQ ID NO: 7)
ttAgATcTCttttgactcatagaggaaagcatcgcggacaaactttttcagtttatttgttggcttaatattctattgttatctttattta
tagatgtttatattgcatgaggtggtttttggagagaagaatgaggaagatgcgtcgagccacagaaacgttagctttacatatagcggag
gtgatgtgaaattaatttacaatagaaataatttacatatcaaacagttagatgctttttgtcgttttttaatatttttatgcttgagaaa
aaatacgtaacttatttatgatatggacagtttggccccaa

Bacterial strains comprising the LON operon expressed the T1P when grown at 37° C., shaking. Unmodified strains (e.g., wild-type bacteria not having a mutated fimS) grown in these growth conditions would not produce T1P.

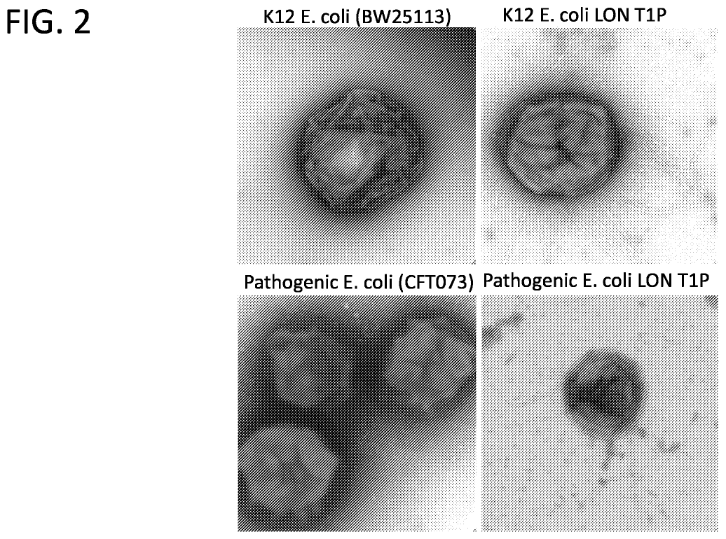

The presence of T1P on the surface of ADAS produced from engineered BW25113 and CFT073 E. coli strains comprising the locked-on operon was confirmed using transmission electron microscopy, as shown in FIG. 2.

### B. ADAS Expressing Heterologous T1P

Heterologous expression of a T1P was achieved by cloning the CFT073 fim operon (ATCC 700928; SEQ ID NO: 1), comprising the genes FimA, Fiml, FimC, FimD, FimF, FimG, and FimH, into a plasmid that contains a constitutive promoter (thus generating pT1P) and expressing the plasmid in a bacterium. The operon was taken from the pathogenic E. coli strain CFT073 and was expressed in the K12 E. coli BW25113. The CFT073 version of the fim operon was chosen due to previous studies showing superiority of the type 1 pilus of this strain in ligand binding. The promoter was J23117 (SEQ ID NO: 2). The bacteria were further engineered to produce ADAS by deleting the minCDE locus using Lambda-RED recombineering, as described in WO 2020/123569.

### Example 2: Endogenous and Heterologous T1P-ADAS Agglutinate Red Blood Cells

To determine whether the engineered bacteria of Example 1 and ADAS produced therefrom comprised T1P at a detectable level, the bacteria and ADAS were assessed using a red blood cell (RBC) hemagglutination assay.

The RBC hemagglutination assay is a standard assay in the field to demonstrate expression of functional T1P. T1P bind mannosylated residues, and guinea pig RBCs (gpRBC) are highly mannosylated. The assay was performed by incubating a dilution series (2x) of the test article (e.g., engineered bacteria of Example 1 or ADAS produced therefrom) in PBS or PBS+mannose with a fixed number of gpRBC and determining the dilution at which the test article no longer agglutinates the RBC (read out as the HA Titer). PBS+mannose was included a functional control, as T1P agglutination is mannose-sensitive.

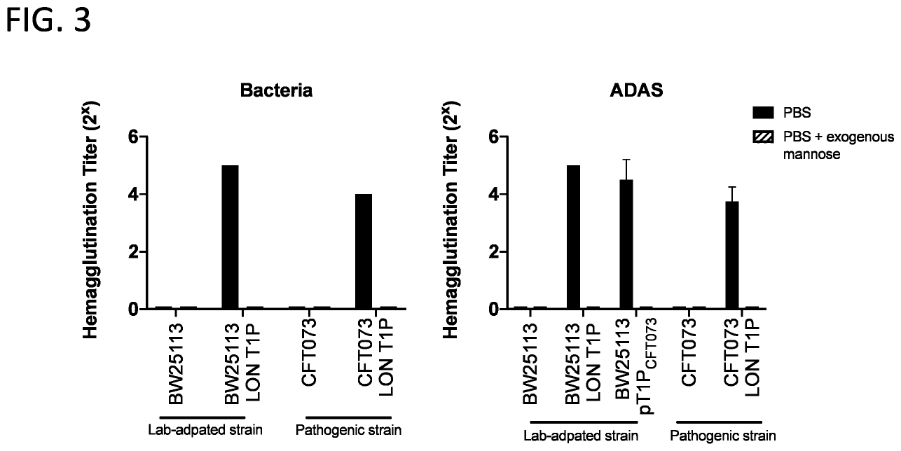

ADAS expressing T1P from endogenous (LON T1P; Example 1A) and heterologous (pT1P; Example 1B) sources were found to agglutinate RBCs (FIG. 3). This binding was mannose-inhibitable, which is the hallmark of T1P-dependent agglutination. Further, functional expression was shown in T1P-ADAS derived from two different E. coli strains (FIG. 3).

### Example 3: Endogenous T1P-ADAS Show Enhanced Binding to Intestinal Cells

Previous research has shown that T1P promote tropism to areas within the intestines (e.g., Peyer's patches) and other body sites and cell types (Martinez et al., The EMBO Journal, 19:2803-2812, 2000; Carvalho et al.; J Exp Med., 206 (10): 2179-2189, 2009; Hase et al., Nature, 462 (7270): 226-230, 2009; Avalos et al., Sci Rep., 6:18109, 2016; Sheikh et al., PLOS Negl Trop Dis., 11 (5): e00055862017, 2017; Spaulding et al., Nature, 546 (7659): 528-532, 2017; Le Guennec et al., Cellular Microbiology, 22: e13132, 2019).

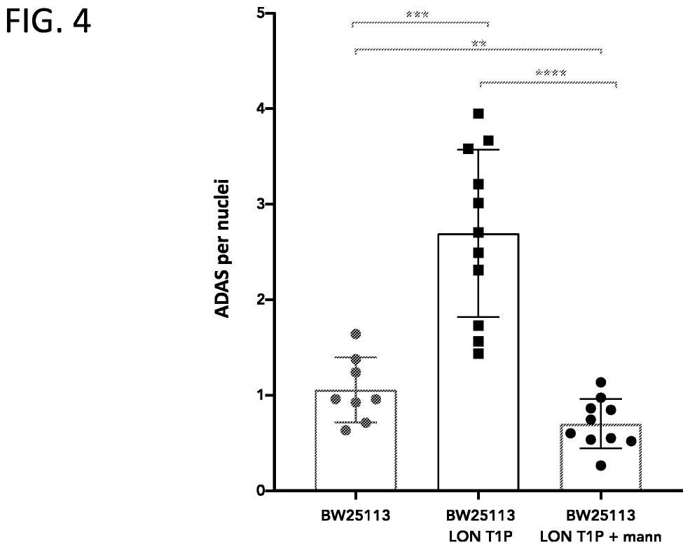

This Example demonstrates that ADAS produced according to Example 1A (T1P-ADAS) show enhanced binding over non-T1P-ADAS (i.e., ADAS produced from bacteria that have not been engineered to comprise a locked-on fim operon according to Example 1A) to the human colorectal cancer cell line HT-29. The assay was performed by incubating ADAS with the HT-29 cells for an hour, removing unbound ADAS, fixing the remaining bound ADAS, and staining with a fluorescent antibody against LPS. Fluorescence microscopy to count the number of ADAS per HT-29 cell nucleus (FIG. 4). It was also demonstrated that the enhanced binding of T1P-ADAS was mannose-sensitive.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

What is claimed is:

1. A preparation comprising a plurality of achromosomal dynamic active systems (ADAS) derived from parent bacterial cells genetically engineered to constitutively express a type 1 pilus (T1P), wherein the plurality of ADAS binds a target cell via the T1P.

2. The preparation of claim 1, wherein the parent bacterial cells comprise a modified fimS promoter that is operably linked to, and directs constitutive expression of, the components of the T1P.

3. The preparation of claim 1 or 2, wherein the components of the T1P are encoded by a fim operon.

4. The preparation of claim 3, wherein the parent bacterial cells comprise a modified fimS promoter operably linked to the fim operon, wherein the modified fimS promoter comprises a mutation at a recombinase cleavage site that prevents recombination of the fimS promoter into an ‘OFF’ orientation.

5. The preparation of any one of claims 1-4, wherein the parent bacterial cells express the components of the T1P at a level that is at least 1.5-fold greater than a level observed in an unmodified parent bacterial cell.

6. The preparation of any one of claims 1-5, wherein the plurality of ADAS comprise the T1P at a level that is at least 1.5-fold greater than a level observed in a plurality of ADAS produced from unmodified parent bacterial cells.

7. The preparation of any one of claims 1-6, wherein the proportion of the plurality of ADAS comprising a T1P is increased relative to a plurality of ADAS produced by parent bacterial cells that do not constitutively express the components of a T1P.

8. The preparation of any one of claims 1-7, wherein the parent bacterial cells comprise an endogenous fim operon.

9. The preparation of any one of claims 1-8, wherein the parent bacterial cells are E. coli bacteria.

10. The preparation of claim 9, wherein the E. coli bacteria are E. coli CFT073.

11. The preparation of any one of claims 1-10, wherein the parent bacterial cells comprise one or more heterologous nucleotide sequences encoding the components of the T1P.

12. The preparation of claim 11, wherein the one or more heterologous nucleotide sequences comprise a fim operon.

13. The preparation of claim 12, wherein the fim operon is the fim operon of E. coli CFT073.

14. The preparation of claim 13, wherein the one or more heterologous nucleotide sequences comprise a sequence having at least 90% identity to the nucleotide sequence of SEQ ID NO: 1.

15. The preparation of claim 14, wherein the one or more heterologous nucleotide sequences comprise a sequence having at least 95% identity to the nucleotide sequence of SEQ ID NO: 1.

16. The preparation of claim 15, wherein the one or more heterologous nucleotide sequences comprise the nucleotide sequence of SEQ ID NO: 1.

17. The preparation of any one of claims 12-16, wherein the one or more heterologous nucleotide sequences further comprise a constitutive promoter operably linked to the fim operon.

18. The preparation of claim 17, wherein the constitutive promoter is a modified fimS promoter comprising a mutation at a recombinase cleavage site that prevents recombination of the fimS promoter into an ‘OFF’ orientation.

19. The preparation of any one of claims 11-18, wherein the one or more nucleotide sequences encoding the components of the T1P are carried on a vector.

20. The preparation of claim 19, wherein the parent bacterial cells have been transiently transformed with the vector.

21. The preparation of claim 19, wherein the parent bacterial cells have been stably transformed with the vector.

22. The preparation of any one of claims 10-21, wherein the parent bacterial cells are Gram-negative bacterial cells.

23. The preparation of claim 22, wherein the Gram-negative bacterial cells are E. coli, Salmonella, Yersinia, Vibrio, Pseudomonas, Shigella, or Legionella bacterial cells.

24. The preparation of any one of claims 11-23, wherein the parent bacterial cells do not comprise a complete endogenous fim operon.

25. The preparation of any one of claims 3, 4, and 12-24, wherein the parent bacterial cells have not been exposed to a culture condition that promotes the expression of the fim operon.

26. The preparation of claim 25, wherein the culture condition is temperature, pH, osmolality, shaking, or activation of the stress or stringent response.

27. The preparation of any one of claims 1-26, wherein the ADAS comprise a cargo.

28. The preparation of claim 27, wherein the cargo is a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP).

29. The preparation of claim 27 or 28, wherein the cargo is encapsulated by the ADAS.

30. The preparation of claim 27 or 28, wherein the cargo is attached to the surface of the ADAS.

31. The preparation of any one of claims 1-30, wherein the ADAS comprise a heterologous bacterial secretion system.

32. The preparation of claim 31, wherein the heterologous bacterial secretion system is a type 3 secretion system (T3SS) or a type 6 secretion system (T6SS).

33. The preparation of claim 31 or 32, wherein the cargo comprises a moiety that directs export by the bacterial secretion system.

34. A composition comprising the preparation of a plurality of ADAS of any one of claims 1-33.

35. The composition of claim 34, wherein the composition is formulated for delivery to a mammal.

36. The composition of claim 35, wherein the composition is formulated for oral delivery.

37. A method for delivering an ADAS to a cell, the method comprising contacting a cell with a composition comprising the preparation of a plurality of ADAS of any of claims 1-36.

38. The method of claim 37, wherein delivery of the ADAS to the cell is increased by at least 10% relative to an ADAS derived from an unmodified parent bacterial cell.

39. The method of claim 37 or 38, wherein an effective amount of the ADAS is delivered to the cell at a dose that is at least 10% lower than the dose required for an ADAS derived from an unmodified parent bacterial cell.

40. A method for delivering a cargo to a cell, the method comprising contacting a cell with a composition comprising the preparation of a plurality of the ADAS of any one of claims 1-36, wherein the ADAS further comprise a cargo.

41. The method of any one of claims 37-40, wherein the ADAS further comprise a heterologous bacterial secretion system.

42. The method of claim 41, wherein the heterologous bacterial secretion system is a T3SS or T6SS.

43. The method of any one of claims 37-42, wherein the delivery is to the cytoplasm of the cell.

44. The method of any one of claims 40-43, wherein delivery of the cargo to the cell is increased by at least 10% relative to an ADAS derived from an unmodified parent bacterial cell.

45. The method of any one of claims 40-43, wherein an effective amount of the cargo is delivered to the cell at a dose that is at least 10% lower than the dose required for an ADAS derived from an unmodified parent bacterial cell.

46. A method of modulating a cell, the method comprising contacting a cell with a composition comprising the preparation of a plurality of ADAS of any one of claims 1-36, whereby the cell is modulated.

47. The method of claim any one of claims 37-46, wherein the cell is a mammalian cell.

48. The method of claim 47, wherein the mammalian cell is a gut cell.

49. The method of claim 48, wherein the gut cell is a gut-associated lymphoid tissue (GALT) cell, a Peyer's patch cell, an M cell, a lamina propria cell, a small intestine cell, or a large intestine cell.

50. The method of claim 47, wherein the mammalian cell is a bladder cell.

51. The method of claim 47, wherein the mammalian cell is an immune cell.

52. The method of claim 47, wherein the mammalian cell is a blood-brain barrier cell.

53. The method of any one of claims 47-52, wherein the mammalian cell is a mannosylated cell.

54. An ADAS derived from a parent bacterial cell genetically engineered to constitutively express a T1P, wherein the ADAS is produced by a process comprising the steps of:
(a) providing a parent bacterial cell that has been modified to constitutively express the components of a T1P; and
(b) producing an ADAS from the parent bacterial cell, wherein the ADAS comprises the T1P.

55. An ADAS derived from a parent bacterial cell genetically engineered to constitutively express a T1P, wherein the ADAS is produced by a process comprising the steps of:
(a) providing a parent bacterial cell that has been modified to express the components of a native T1P at a level that is at least 1.5-fold greater than a level observed in an unmodified parent bacterial cell; and
(b) producing an ADAS from the parent bacterial cell, wherein the ADAS comprises the T1P that is native to the parent bacterial cell.

56. An ADAS derived from a parent bacterial cell genetically engineered to constitutively express a T1P, wherein the ADAS is produced by a process comprising the steps of:
(a) providing a parent bacterial cell that has been modified to express the components of a heterologous T1P; and
(b) producing an ADAS from the parent bacterial cell, wherein the ADAS comprises the T1P that is heterologous to the parent bacterial cell.

57. A genetically engineered bacterium that constitutively expresses the components of a T1P, wherein the engineered bacterium comprises the T1P at a level which is at least 1.5-fold greater compared to the level of T1P comprised by a non-engineered bacterium.

58. The bacterium of claim 57, wherein the T1P is a native T1P.

59. The bacterium of claim 57, wherein the T1P is a heterologous T1P.

60. A genetically engineered bacterium that constitutively expresses the components of a T1P, wherein the bacterium has been modified to produce ADAS.

61. A method for producing an ADAS, the method comprising producing an ADAS from a bacterium genetically engineered to constitutively expresses the components of a T1P.

62. An ADAS produced according to the method of claim 61.
